(19) United States
(12) Patent Application Publication
(10) Pub. No.: US 2022/0065863 A1
(43) Pub. Date: Mar. 3, 2022

(54) PREDICTIVE BIOMARKERS FOR ADVERSE EFFECTS OF RADIATION THERAPY

(71) Applicant: Georgetown University, Washington, DC (US)

(72) Inventors: Anatoly Dritschilo, Bethesda, MD (US); Amrita K. Cheema, Potomac, MD (US); Scott Grindrod, Falls Church, VA (US); Xiaogang Zhong, Greenbelt, MD (US)

(73) Assignee: Georgetown University, Washington, DC (US)

(21) Appl. No.: 17/476,148

(22) Filed: Sep. 15, 2021

Related U.S. Application Data
(63) Continuation-in-part of application No. 16/771,786.
(60) Provisional application No. 62/597,172, filed on Dec. 11, 2017.

(51) Int. Cl. G01N 33/574; A61N 5/10

(52) U.S. Cl. CPC G01N 33/57484; A61N 5/10

(57) ABSTRACT

Methods of treating with radiation therapy a subject having cancer, in which the method comprises administering radiation therapy to the subject. The subject does not have an increased risk of having an adverse reaction to radiation therapy. The subject has an increased risk of an adverse reaction to radiation therapy when the subject's level of each component in a component profile from a sample of the subject is altered as compared to the normal level of each component. The component profile may comprise a metabolite panel of geranyl pyrophosphate, glucose-1-phosphate, and 3-hydroxy-3-methylglutaryl-CoA; a lipid panel of LPA 18:0, LPA 16:0, LPC 20:2, CER 24:0, and LPI 16:1; or a combination of these panels. The component profile may also comprise a metabolite panel of metanephrine, tryptophan, xanthurenic acid, and pantothenate; a lipid panel of LPA 18:0, DAG 16:0/18:0, LPA 16:0, and DAG 18:1/18:1; or a combination of these panels.

### CROSS-REFERENCE TO RELATED APPLICATIONS

This application is a continuation-in-part of U.S. application Ser. No. 16/771,786 filed on Jun. 11, 2020, which is a National Stage of International Application No. PCT/US2018/064924 filed on Dec. 11, 2018, which claims the benefit of priority to U.S. Provisional Application No. 62/597,172 filed on Dec. 11, 2017, all of which are hereby incorporated by reference in their entireties or all purposes.

### STATEMENT REGARDING FEDERALLY SPONSORED RESEARCH OR DEVELOPMENT

This invention was made with Government support under grant no. HHSN261201600027C awarded by National Institutes of Health. The government has certain rights in the invention.

### FIELD OF INVENTION

The present invention relates to methods of determining if a subject has an increased risk of having an adverse reaction to receiving radiation therapy for cancer, for example prostate cancer. The methods comprise analyzing at least one sample from the subject to determine a value of the subject's metabolite profile, and comparing the value of the subject's metabolite profile with the value obtained from subjects determined to define a normal metabolite profile, to determine if the subject's metabolite profile is altered compared to a normal metabolite profile. A difference in the value of the subject's metabolite profile compared to those defined as having a normal metabolite profile is indicative that the subject has an increased risk of having an adverse reaction to receiving radiation therapy for cancer, for example prostate cancer

### BACKGROUND OF THE INVENTION

Radiation therapy (RT) is an effective modality as a primary treatment of cancers, or as an adjuvant to surgery or chemotherapy. Risks, benefits, and late effects of radiation therapy are observed in the heterogeneous clinical responses of patients receiving curative radiation therapy. In principle, all cancers can be controlled if sufficient radiation doses can be delivered to tumors; however, in practice, the achievable radiation doses are frequently limited by toxicities that may result following exposure of normal tissues to high radiation doses. Organ-specific tissue injuries following prostate irradiation may include acute toxicities (such as cystitis and enteritis), late toxicities (such as rectal of bladder bleeding) and broad toxicities such as bone marrow depletion or soft tissue necrosis. Strategies to improve the therapeutic index of RT have focused on conformal technologies to target tumors more exactly, limit the volume of exposed normal tissues and limit the doses delivered to normal tissues. The technology includes computer assisted shaping of the radiation doses; examples include intensity modulated radiation therapy (IMRT), stereotactic radiosurgery (SRS) and high-dose rate (HDR) brachytherapy, as well as the use of particle therapy (proton beam therapy (PBT) or carbon ions). Despite the sophistication of current technologies, the need to deliver high doses of radiation to tumors results in normal tissue toxicities in subsets of patients.

Variations in patients' normal tissue sensitivities to radiation have been attributed to genetic factors, including mutations in genes associated with DNA repair processes, immunological diseases, and connective tissue diseases. Extreme examples are provided by the genetic syndromes of ataxia-telangiectasia, Nijmegen breakage syndrome and the clinical syndromes of scleroderma and systemic Lupus erythematosus.

The past decade has seen major developments in the treatment of cancer, including technical improvements in radiotherapy. A fraction of patients treated for cancer, however, experience radiation treatment related acute and late effects that adversely affect quality of life and also lead to tumor recurrence episodes. The manifestation of these symptoms takes months to develop and raises an urgent need for developing smarter strategies for symptom anticipation and management. Application of personalized medicine for patient treatment has further highlighted the need for clinical biomarkers to predict response and to direct therapy.

Prostate cancer patients that are susceptible to radiation induced adverse effects carry a biochemical fingerprint that could be characterized using blood based metabolomics. Furthermore, these molecular changes may provide insight into specific pathway perturbations that could be used to instruct clinical therapeutics. Based on a retrospective outcome study, a biomarker panel was delineated for prediction of radiation response in patients treated for prostate cancer. Such biomarkers may aid in early detection of tissue toxicity in cancer patients, informing clinical decisions for treatment and follow-up management in patients at risk.

### SUMMARY OF THE INVENTION

Some of the main aspects of the present invention are summarized below. Additional aspects are described in the Detailed Description of the Invention, Example, and Claims sections of this disclosure. The description in each section of this disclosure is intended to be read in conjunction with the other sections. Furthermore, the various embodiments described in each section of this disclosure can be combined in various ways, and all such combinations are intended to fall within the scope of the present invention.

Accordingly, one aspect of the invention relates to a method of treating with radiation therapy a subject having cancer. The method comprises administering radiation therapy to the subject, in which the subject does not have an increased risk of having an adverse reaction to radiation therapy. The subject has an increased risk of an adverse reaction to radiation therapy when the subject's level of each component in a component profile from a sample of the subject is altered as compared to a normal level of each component.

The adverse reaction may be tumor recurrence, or may be one or more late effects. In some embodiments, the late effects may comprise rectal toxicity, urinary toxicity, or a combination thereof.

In some embodiments where the adverse reaction is tumor recurrence, the component profile may comprise geranyl pyrophosphate, glucose-1-phosphate, and 3-hydroxy-3-methylglutaryl-CoA. In certain embodiments, the subject's level of each component is altered as compared to the normal level of each component when the subject's level of geranyl pyrophosphate, glucose-1-phosphate, and 3-hydroxy-3-methylglutaryl-CoA is higher as compared to the normal level of geranyl pyrophosphate, glucose-1-phosphate, and 3-hydroxy-3-methylglutaryl-CoA, respectively.

In some embodiments where the adverse reaction is tumor recurrence, the component profile may comprise lysophosphatidic acid (LPA) 18:0, LPA 16:0, lysophosphatidylcholine (LPC) 20:2, ceramide (CER) 24:0, and lysophosphatidylinositol (LPI) 16:1. In certain embodiments, the subject's level of each component is altered as compared to the normal level of each component when the subject's level of LPA 18:0, LPA 16:0, and LPC 20:2 is higher than the normal level of LPA 18:0, LPA 16:0, and LPC 20:2, respectively; and the subject's level of CER 24:0 and LPI 16:1 is lower than the normal level of CER 24:0 and LPI 16:1, respectively.

In some embodiments where the adverse reaction is tumor recurrence, the component profile may comprise geranyl pyrophosphate, glucose-1-phosphate, 3-hydroxy-3-methylglutaryl-CoA, LPA 18:0, LPA 16:0, LPC 20:2, CER 24:0, and LPI 16:1. In certain embodiments, the subject's level of each component is altered as compared to the normal level of each component when the subject's level of geranyl pyrophosphate, glucose-1-phosphate, 3-hydroxy-3-methylglutaryl-CoA, LPA 18:0, LPA 16:0, and LPC 20:2 is higher as compared to the normal level of geranyl pyrophosphate, glucose-1-phosphate, 3-hydroxy-3-methylglutaryl-CoA, LPA 18:0, LPA 16:0, and LPC 20:2, respectively; and the subject's level of CER 24:0 and LPI 16:1 is lower than the normal level of CER 24:0 and LPI 16:1, respectively.

In some embodiments where the adverse reaction is one or more late effects, the component profile may comprise metanephrine, tryptophan, xanthurenic acid, and pantothenate. In certain embodiments, the subject's level of each component is altered as compared to the normal level of each component when the subject's level of metanephrine, tryptophan, xanthurenic acid, and pantothenate is lower than the normal level of metanephrine, tryptophan, xanthurenic acid, and pantothenate, respectively.

In some embodiments where the adverse reaction is one or more late effects, the component profile may comprise LPA 18:0, diacylglycerol (DAG) 16:0/18:0, LPA 16:0, and DAG 18:1/18:1. In certain embodiments, the subject's level of each component is altered as compared to the normal level of each component when the subject's level of LPA18:0 and LPA 16:0 is higher than the normal level of LPA18:0 and LPA 16:0, respectively; and the subject's level of DAG 16:0/18:1 and DAG 18:1/18:1 is lower than the normal level of DAG 16:0/18:1 and DAG 18:1/18:1, respectively.

In some embodiments where the adverse reaction is one or more late effects, the component profile may comprise metanephrine, tryptophan, xanthurenic acid, pantothenate, LPA 18:0, DAG 16:0/18:0, LPA 16:0, and DAG 18:1/18:1. In certain embodiments, the subject's level of each component is altered as compared to the normal level of each component when the subject's level of LPA18:0 and LPA 16:0 is higher than the normal level of LPA18:0 and LPA 16:0, respectively; and the subject's level of metanephrine, tryptophan, xanthurenic acid, pantothenate, DAG 16:0/18:1, and DAG 18:1/18:1 is lower than the normal level of metanephrine, tryptophan, xanthurenic acid, pantothenate, DAG 16:0/18:1, and DAG 18:1/18:1, respectively.

The normal level of the component may comprise the subject's level of the component prior to receiving radiation treatment for cancer. Alternatively, the normal level of the component may comprise a level generated from a population of individuals that did not have an adverse reaction after receiving radiation treatment for cancer.

Another aspect of the invention relates to a method of measuring levels of components in a component profile in a subject. The method may comprise determining the level of each of the components.

In some embodiments, the component profile comprises geranyl pyrophosphate, glucose-1-phosphate, and 3-hydroxy-3-methylglutaryl-CoA.

In some embodiments, the component profile comprises LPA 18:0, LPA 16:0, LPC 20:2, CER 24:0, and LPI 16:1.

In some embodiments, the component profile comprises geranyl pyrophosphate, glucose-1-phosphate, 3-hydroxy-3-methylglutaryl-CoA, LPA 18:0, LPA 16:0, LPC 20:2, CER 24:0, and LPI 16:1

In some embodiments, the component profile comprises metanephrine, tryptophan, xanthurenic acid, and pantothenate.

In some embodiments, the component profile comprises LPA 18:0, DAG 16:0/18:0, LPA 16:0, and DAG 18:1/18:1.

In some embodiments, the component profile comprises metanephrine, tryptophan, xanthurenic acid, pantothenate, LPA 18:0, DAG 16:0/18:0, LPA 16:0, and DAG 18:1/18:1.

In some embodiments, the method further comprises obtaining the sample from the subject.

Further aspects, features, and advantages of the present invention will be better appreciated upon a reading of the following detailed description of the invention and claims.

### BRIEF DESCRIPTION OF THE DRAWINGS

FIG. 1 depicts predictive biomarkers of recurrence episodes in prostate cancer as described in Example 2B. Panel A shows a receiver operating characteristic (ROC) curve for an eight-metabolite panel for classification of patients with recurrence episodes. Panel B shows the eight-metabolite panel predictive of recurrence.

FIG. 2 depicts box and whisker plots of the plasma eight-metabolite panel results for normal and recurrence groups in the prostate cancer cohort, as described in Example 2B.

FIG. 3 depicts plasma metabolite index (PMI) plot representation demonstrating group stratification, as described in Example 2B. The PMI results are based on the logistic regression model are illustrated as a boxplot that distinguishes between prostate cancer patients who developed recurrence and those that remained cancer free post stereotactic body radiation therapy (SBRT). Solid black horizontal lines represent the mean value, while the whiskers denote the spread within a group. Orange and light blue dots represent PC patients who received hormone therapy or not, respectively. The higher index values (left vertical axis) are associated with an increased risk of recurrence in the prostate cancer cohort. The confidence interval (right vertical axis) of predicting risk of recurrence, transitions from 90 to 100% at a relative index value of 2.

FIG. 4 depicts predictive biomarkers of rectal toxicity episodes, post SBRT in the prostate cancer cohort, as described in Example 2C. Panel A shows ROC curve for a six-metabolite panel for classification of patients with radiation proctitis. Panel B shows the six-metabolite panel predictive of rectal toxicity.

FIG. 5 depicts box and whisker plots of plasma six-metabolite panel results for normal and radiation proctitis groups in the prostate cancer cohort, as described in Example 2C.

FIG. 6 depicts PMI plot representation demonstrating group stratification, as described in Example 2C. The PMI results are based on the logistic regression model that are illustrated as a boxplot that distinguishes between prostate cancer patients who developed rectal toxicity and those that remained normal, post SBRT. Solid black horizontal lines represent the mean value, while the whiskers denote the spread within a group. Orange and light blue dots represent PC patients who received hormone therapy or not, respectively. The higher index values (left vertical axis) are associated with an increased risk of radiation proctitis (RP) in the prostate cancer cohort. The confidence interval (right vertical axis) of predicting risk of RP, transitions from 90 to 100% at a relative index value of 5.

FIG. 7 depicts predictive biomarkers of urinary toxicity episodes, post SBRT in the prostate cancer cohort, as described in Example 2D. Panel A shows ROC curve for a nine-metabolite panel for classification of patients with urinary toxicity episodes. Panel B shows the nine-metabolite panel predictive of urinary toxicity.

FIG. 8 depicts box and whisker plots of nine-plasma metabolite panel results for normal and group experiencing urinary toxicity in the prostate cancer cohort, as described in Example 2D.

FIG. 9 depicts PMI plot representation demonstrating group stratification, as described in Example 2D. The PMI results are based on the logistic regression model are illustrated as a boxplot that distinguishes between prostate cancer patients who developed urinary toxicity and those that remained normal post SBRT. Solid black horizontal lines represent the mean value, while the whiskers denote the spread within a group. Orange and light blue dots represent PC patients who received hormone therapy or not, respectively. The higher index values (left vertical axis) are associated with an increased risk of urinary toxicity in the prostate cancer cohort. The confidence interval (right vertical axis) of predicting risk of recurrence, transitions from 90 to 100% at a relative index value of 7.5.

FIG. 10 shows ROC curve for a three-metabolite panel for classification of patients with tumor recurrence, as described in Example 3.

FIG. 11 shows ROC curve for a four-metabolite panel for classification of patients with late effects, as described in Example 3.

FIG. 12 shows ROC curve for a five-lipid panel for classification of patients with tumor recurrence, as described in Example 3.

FIG. 13 shows ROC curve for a four-lipid panel for classification of patients with late effects, as described in Example 3.

FIG. 14 shows ROC curve for a three-metabolite, five-lipid panel for classification of patients with tumor recurrence, as described in Example 3.

FIG. 15 shows ROC curve for a four-metabolite, four-lipid panel for classification of patients with tumor recurrence, as described in Example 3.

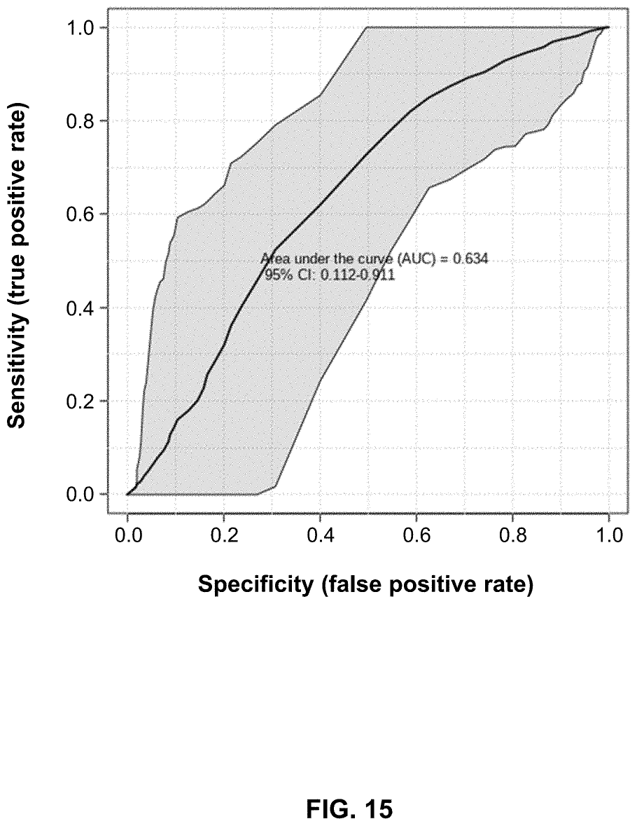

### DETAILED DESCRIPTION OF THE INVENTION

The practice of the present invention will employ, unless otherwise indicated, conventional techniques of pharmaceutics, formulation science, oncology, immunology, hematology, cell biology, molecular biology, clinical pharmacology, and clinical practice, which are within the skill of the art.

In order that the present invention can be more readily understood, certain terms are first defined. Additional definitions are set forth throughout the disclosure. Unless defined otherwise, all technical and scientific terms used herein have the same meaning as commonly understood by one of ordinary skill in the art to which this invention is related.

Any headings provided herein are not limitations of the various aspects or embodiments of the invention, which can be had by reference to the specification as a whole. Accordingly, the terms defined immediately below are more fully defined by reference to the specification in its entirety.

All references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

The phraseology or terminology in this disclosure is for the purpose of description and not of limitation, such that the terminology or phraseology of the present specification is to be interpreted by the skilled artisan in light of the teachings and guidance.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Units, prefixes, and symbols are denoted in their Systeme International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value encompassed by the range. For example, a stated range of 5-10 is also a disclosure of 5, 6, 7, 8, 9, and 10. Where a numeric term is preceded by “about,” the term includes the stated number and values ±10% of the stated number.

### Methods of the Invention

The present invention relates to methods of treating with radiation therapy a subject having cancer.

In embodiments of the invention, the methods comprise (a) determining whether the subject has an increased risk of having an adverse reaction to radiation therapy by (i) analyzing at least one sample from the subject to determine the subject's level of each component in a component profile, and (ii) comparing the subject's level of each component in the component profile to a normal level of each component, wherein the subject is determined to have an increased risk of an adverse reaction to radiation therapy when the subject's level of each component is altered as compared to the normal level of each component; and (b) administering radiation therapy to the subject when it is determined that the subject does not have an increased risk of having an adverse reaction to radiation therapy.

In embodiments of the invention, the methods comprise (a) monitoring whether the subject has an increased risk of having an adverse reaction to radiation therapy, in which the monitoring comprises, at two or more time points, (i) analyzing at least one sample from the subject to determine the subject's level of each component in a component profile, and (ii) comparing the subject's level of each component in the component profile to a normal level of each component, wherein the subject is determined to have an increased risk of an adverse reaction to radiation therapy when the subject's level of each component is altered as compared to the normal level of each component at at least two timepoints; and (b) administering radiation therapy to the subject when it is determined that the subject does not have an increased risk of having an adverse reaction to radiation therapy.

In embodiments of the invention, the methods comprise administering radiation therapy to a subject that does not have an increased risk of having an adverse reaction to radiation therapy. In some embodiments, the subject has an increased risk of having an adverse reaction to radiation therapy when the subject's level of each component in a component profile is altered as compared to the normal level of each component. In these embodiments, the subject does not have an increased risk of having an adverse reaction to radiation therapy when it is not the case that the subject's level of each component in a component profile is altered as compared to the normal level of each component.

As used herein, the term subject or “test subject” indicates a mammal, in particular a human or non-human primate. As used herein, the phrase cancer is understood in the art and is used to mean such things as an abnormal growth, such as hyperplasia, neoplasia, or a tumor, to name a few. According to the methods of the present invention, the subject having the cancer would be a candidate for radiation therapy. Thus, the cancer as used herein does not include those cancers for which radiation therapy is not an option. In one embodiment, the cancer is prostate cancer. As used herein, the phrase “prostate cancer” is well-understood in the art and means a cancer in the prostate gland. Prostate cancer can be characterized according to the Gleason score, which is a system based on five distinct patterns in which prostate cancer cells can be grouped as they change from normal cells to tumor cells. As used herein, “prostate cancer” can be a pre-cancerous lesion with a low Gleason score, including lesions having a Grade 1, Grade 2, Grade 3, Grade 4, Grade 5, Grade 6, Grade 7, Grade 8, Grade 9, or Grade 10 Gleason score. In addition, “prostate cancer” includes Stage 1, Stage 2, Stage 3, Stage 4, or even recurrent prostate cancer.

As used herein, the term “adverse reaction” as it relates to radiation therapy for cancer is cancer or tumor recurrence after receiving the therapy or a late effect. As used herein, the recurrence of cancer, for example prostate cancer, can occur any time after the subject receives the therapy and would be considered in remission or even “cured.” As used herein, a subject is “cured” of cancer, for example prostate cancer, at least initially, if the subject is in remission and remains cancer free for at least 5 years from the time of the prescribed therapy to remove or destroy the diseased tissue. As used herein, “late effect” refers to a side effect of cancer treatment that become apparent after treatment has concluded. Examples of late effects include, but are not limited, to rectal toxicity and urinary toxicity.

As used herein, the rectal toxicity should be attributable to the subject receiving radiation therapy for prostate cancer. As is well-understood, rectal toxicity can occur as a result of other factors besides receiving radiation. Thus, the methods of the present invention include determining if a subject has or has a risk of developing rectal toxicity from a source other than receiving radiation therapy for cancer, for example prostate cancer. If the subject is free of rectal toxicity prior to receiving radiation therapy for cancer, the methods of the present invention can be performed on the subject that is a candidate for receiving radiation therapy for cancer.

As used herein, the urinary toxicity should be attributable to the subject receiving radiation therapy for cancer. As is well-understood, urinary toxicity can occur as a result of other factors besides receiving radiation. Thus, the methods of the present invention include determining if a subject has or has a risk of developing urinary toxicity from a source other than receiving radiation therapy for cancer. If the subject does not have urinary toxicity prior to receiving radiation therapy for cancer, the methods of the present invention can be performed on the subject that is a candidate for receiving radiation therapy for cancer, for example prostate cancer.

If it is determined that a subject has an increased risk of an adverse reaction to radiation therapy for cancer, the attending health care provider may subsequently prescribe or institute a treatment program or prescribe a different treatment for cancer. In this manner, the present invention also provides for methods of screening individuals as candidates for treatment of an adverse reaction to radiation therapy for cancer. The attending healthcare worker may begin treatment, based on the subject's metabolite profile, before there are perceivable, noticeable, or measurable signs of an adverse reaction to radiation therapy for cancer in the individual.

Similarly, the invention provides methods of treating a subject having cancer, for example prostate cancer. The treatment methods include obtaining a subject's component or composite profile as defined herein and prescribing a treatment regimen to the subject if the component and/or composite profile indicate that the subject is at risk of suffering from an adverse reaction to radiation therapy for cancer, for example prostate cancer.

Suitable radiation therapies for prostate cancer are well-known, and the methods disclosed and described herein are not dependent on the specific type of radiation therapy for cancer.

In embodiments of the invention in which the method of treatment includes monitoring whether the subject has an increased risk of having an adverse reaction to radiation therapy, or in which the determination that the subject does not have an increased risk of having an adverse reaction to radiation therapy involved monitoring whether the subject has an increased risk of having an adverse reaction to radiation therapy, the analysis of at least one sample from the subject to determine the subject's level of each component in a component profile, and the comparison of the subject's level of each component in the component profile to a normal level of each component may occur between about once and 20 times in a year, or between about once and 15 times in a year, or between about once and about ten times in a year, or between about once and five times in a year, including once, twice, three times, four times, five times, six times, seven times, eight times, nine times, 10 times, 11 times, 12 times, 13 times, 14 times, 15 times, 16 times, 17 times, 18 times, 19 times, or 20 times in a year; these frequencies can also serve as endpoints for a range of frequencies at which the comparison and analysis can occur in a year, for example, about two times to about five times, about two times to three times, etc. The analysis and comparison may also occur for one or more years, such as between about one year and 20 years, or between about one year and 15 years, or between about one year and ten years, or between about one year and five years, including for one year, two years, three years, four years, five years, six years, seven years, eight years, nine years, 10 years, 11 years, 12 years, 13 years, 14 years, 15 years, 16 years, 17 years, 18 years, 19 years, or 20 years; these durations can also serve as endpoints for a range of durations over which the analysis and comparisons can occur, for example, about two years to ten years, about three years to five years, etc.

In some embodiments, the subject is determined to have an increased risk of an adverse reaction to radiation therapy when the subject's level of each component is altered as compared to the normal level of each component at between about two time points and 20 time points, or between about two time points and 15 time points, or between about two time points and ten time points, or between about two time points and five time points, or between about two time points and four time points, or between about two time points and three time points, including about two time points, three time points, four time points, five time points, six time points, seven time points, eight time points, nine time points, 10 time points, 11 time points, 12 time points, 13 time points, 14 time points, 15 time points, 16 time points, 17 time points, 18 time points, 19 time points, or 20 time points; these time points can also serve as endpoints for a range of time points, for example, about three time points to six time points, about four time points to five time points, etc. These time points may be over a period of about one year to 20 years, or about one year to 15 years, or about one year to ten years, or about one year to five years, or about one year to four years, or about one year to three years, or about one year to two years, including one year, two years, three years, four years, five years, six years, seven years, eight years, nine years, 10 years, 11 years, 12 years, 13 years, 14 years, 15 years, 16 years, 17 years, 18 years, 19 years, or 20 years; these durations can also serve as endpoints for a range of durations, for example, about two years to five years, about two years to three years, etc.

### Determination of Increased Risk

As used herein, the term “increased risk” is used to mean that the test subject has an increased chance of developing or acquiring an adverse reaction to radiation therapy for cancer, for example prostate cancer, compared to a normal individual. The increased risk may be relative or absolute and may be expressed qualitatively or quantitatively. For example, an increased risk may be expressed as simply determining the subject's component profile and placing the patient in an “increased risk” category, based upon previous population or individual studies. Alternatively, a numerical expression of the subject's increased risk may be determined based upon the component profile. As used herein, examples of expressions of an increased risk include but are not limited to, odds, probability, odds ratio, p-values, attributable risk, metabolite index score, relative frequency, positive predictive value, negative predictive value, and relative risk.

For example, the correlation between a subject's component profile and the likelihood of suffering from an adverse reaction to radiation therapy for cancer may be measured by an odds ratio (OR) and by the relative risk (RR). If P(R⁺) is the probability of developing an adverse reaction to radiation therapy for cancer for individuals with the risk profile (R) and P(R⁻) is the probability of developing an adverse reaction to radiation therapy for cancer for individuals without the risk profile, then the relative risk is the ratio of the two probabilities: RR=P(R⁺)/P(R⁻).

In case-control studies, however, direct measures of the relative risk often cannot be obtained because of sampling design. The odds ratio allows for an approximation of the relative risk for low-incidence diseases and can be calculated: OR=(F⁺/(1−F⁺))/(F⁻/(1−F⁻)), where F⁺ is the frequency of a risk profile in cases studies and F⁻ is the frequency of risk profile in controls. F⁺ and F⁻ can be calculated using the metabolite profile frequencies of the study.

The attributable risk (AR) can also be used to express an increased risk. The AR describes the proportion of individuals in a population exhibiting an adverse reaction to radiation therapy for cancer due to a specific member of the component risk profile. AR may also be important in quantifying the role of individual components (specific member) in reaction etiology and in terms of the public health impact of the individual marker. The public health relevance of the AR measurement lies in estimating the proportion of cases of an adverse reaction to radiation therapy for cancer in the population that could be prevented if the profile or individual component were absent. AR may be determined as follows: AR=P_{E}(RR−1)/(P_{E}(RR−1)+1), where AR is the risk attributable to a profile or individual component of the profile, and PE is the frequency of exposure to a profile or individual component of the profile within the population at large. RR is the relative risk, which can be approximated with the odds ratio when the profile or individual component of the profile under study has a relatively low incidence in the general population.

In one embodiment, the increased risk of a patient can be determined from p-values that are derived from association studies. Specifically, associations with specific profiles can be performed using regression analysis by regressing the component profile with an adverse reaction to radiation therapy for cancer. In addition, the regression may or may not be corrected or adjusted for one or more factors. The factors for which the analyses may be adjusted include, but are not limited to age, sex, weight, ethnicity, geographic location, fasting state, general health of the subject, alcohol or drug consumption, caffeine or nicotine intake and circadian rhythms, and the subject's Prostate Specific Antigen (PSA) status to name a few.

Increased risk can also be determined from p-values that are derived using logistic regression. Binomial (or binary) logistic regression is a form of regression which is used when the dependent is a dichotomy and the independents are of any type. Logistic regression can be used to predict a dependent variable on the basis of continuous and/or categorical independents and to determine the percent of variance in the dependent variable explained by the independents; to rank the relative importance of independents; to assess interaction effects; and to understand the impact of covariate control variables. Logistic regression applies maximum likelihood estimation after transforming the dependent into a “logit” variable (the natural log of the odds of the dependent occurring or not). In this way, logistic regression estimates the probability of a certain event occurring. These analyses are conducted with the program SAS.

SAS (“statistical analysis software”) is a general purpose package (similar to Stata and SPSS) created by Jim Goodnight and N.C. State University colleagues. Ready-to-use procedures handle a wide range of statistical analyses, including but not limited to, analysis of variance, regression, categorical data analysis, multivariate analysis, survival analysis, psychometric analysis, cluster analysis, and nonparametric analysis.

Accordingly, the present invention also relates to methods of determining if a subject has an increased risk of having an adverse reaction to receiving radiation therapy for cancer, for example, prostate cancer. The methods comprise analyzing at least one sample from the subject to determine a value of the subject's component profile, and comparing the value of the subject's component profile with the value obtained from subjects determined to define a normal component profile, to determine if the subject's component profile is altered compared to a normal component profile. A difference in the value of the subject's component profile compared to those defined as having a normal component profile is indicative that the subject has an increased risk of having an adverse reaction to receiving radiation therapy for cancer.

In addition, select embodiments of the present invention comprise the use of a computer comprising a processor and the computer is configured or programmed to generate one or more component profiles and/or to determine statistical risk. The methods may also comprise displaying the one or more profiles and/or risk profiles on a screen that is communicatively connected to the computer. In another embodiment, two different computers can be used: one computer configured or programmed to generate one or more metabolite profiles and a second computer configured or programmed to determine statistical risk. Each of these separate computers can be communicatively linked to its own display or to the same display.

In some embodiments, determining whether the subject has an increased risk of having an adverse reaction to radiation therapy comprises (i) analyzing at least one sample from the subject to determine the subject's level of each component in a component profile, and (ii) comparing the subject's level of each component in the component profile to a normal level of each component, wherein the subject is determined to have an increased risk of an adverse reaction to radiation therapy when the subject's level of each component is altered as compared to the normal level of each component.

Examples of test samples or sources of components for the component profiles include, but are not limited to, biological fluids, which can be tested by the methods of the present invention described herein, and include but are not limited to whole blood, such as but not limited to peripheral blood, serum, plasma, cerebrospinal fluid, urine, semen, lymph fluids, and various external secretions of the respiratory, intestinal and genitourinary tracts, tears, saliva, milk, white blood cells, myelomas and the like. Test samples to be assayed also include but are not limited to tissue specimens including normal and abnormal tissue.

Techniques to assay levels of individual components of the profiles from test samples are well known to the skilled technician, and the invention is not limited by the means by which the components are assessed. Techniques to assay levels of individual components of any non-lipid component of the component profile from test samples are well known to the skilled technician, and the invention is not limited by the means by which the components are assessed. In one embodiment, levels of the individual components of the non-lipid portion of the profile are assessed using quantitative arrays, PCR, Northern Blot analysis, Western Blot analysis, mass spectroscopy, high-performance liquid chromatography (HPLC, high performance gas chromatography (HPGC) and the like. Other methods of assessing levels of the individual components include biological methods, such as but not limited to ELISA assays. To determine levels of metabolites, it is not necessary that an entire metabolite, e.g., a full length protein or an entire RNA transcript, be present or fully sequenced. In other words, determining levels of, for example, a fragment of protein being analyzed may be sufficient to conclude or assess that an individual component of the metabolite profile, including the lipid and non-lipid portions of the metabolite profile, being analyzed is increased or decreased. Similarly, if, for example, arrays or blots are used to determine metabolite levels, the presence/absence/strength of a detectable signal may be sufficient to assess levels of metabolites.

Levels of the individual lipids of the component profile may be assessed using mass spectrometry in conjunction with ultra-performance liquid chromatography (UPLC), high-performance liquid chromatography (HPLC), gas chromatography (GC), gas chromatography/mass spectroscopy (GC/MS), and UPLC to name a few. Other methods of assessing levels of the individual components include biological methods, such as but not limited to ELISA assays.

To this end, an aspect of the present invention relates to methods of measuring levels of components om a subject. The method comprise obtaining a sample from the subject and determining levels of components in the subject's component profile.

The assessment of the levels of the individual components of the metabolite and/or lipid profile can be expressed as absolute or relative values and may or may not be expressed in relation to another component, a standard an internal standard or another molecule of compound known to be in the sample. If the levels are assessed as relative to a standard or internal standard, the standard may be added to the test sample prior to, during or after sample processing.

To assess levels of the individual components of the metabolite and/or lipid profile, a sample is taken from the subject. The sample may or may not be processed prior assaying levels of the components of the metabolite and/or lipid profile. For example, whole blood may be taken from an individual and the blood sample may be processed, e.g., centrifuged, to isolate plasma or serum from the blood. The sample may or may not be stored, e.g., frozen, prior to processing or analysis.

The subject's component profile is compared to the profile that is deemed to be a normal component profile. To establish the component profile of a normal individual, an individual or group of individuals may be first assessed for the lack of any observable or noticeable adverse reactions to radiation therapy for cancer, for example prostate cancer. Once established, the component profile of the individual or group of individuals can then be determined to establish a “normal component profile.” In one embodiment, a normal component profile can be ascertained from the same subject when the subject is deemed to not have cancer, for example prostate cancer, and is displaying no signs (clinical or otherwise) of cancer. In one embodiment, a “normal” component profile is assessed in the same subject from whom the sample is taken prior to the onset of measurable, perceivable, or diagnosed sign of cancer. That is, the term “normal” with respect to a component profile can be used to mean the subject's baseline component profile prior to the onset of cancer or receiving radiation therapy for cancer. The component profile can then be reassessed periodically and compared to the subject's baseline component profile.

In another embodiment, a normal component profile is assessed in a sample from a different subject or patient (from the subject being analyzed) and this different subject does not have or is not suspected of having cancer or showed no observable adverse reaction to receiving radiation therapy for cancer. In still another embodiment, the normal component profile is assessed in a population of healthy individuals, the constituents of which do not have cancer or showed no observable adverse reaction to receiving radiation therapy for cancer. Thus, the subject's component profile can be compared to a normal component profile generated from a single normal sample or a component profile generated from more than one normal sample.

Measurements of the individual components, e.g., level, ratio, log ratios etc., of the normal component profile can fall within a range of values, and values that do not fall within this “normal range” are said to be outside the normal range. These measurements may or may not be converted to a value, number, factor or score as compared to measurements in the “normal range.” For example, a measurement for a specific component that is below the normal range, may be assigned a value or −1, −2, −3, etc., depending on the scoring system devised.

In one embodiment, the “component profile value” can be a single value, number, factor, or score given as an overall collective value to the individual molecular components of the profile, or to the categorical components, i.e., a phosphatidylcholine portion, a biogenic amine portion and/or an amino acid portion. For example, if each component is assigned a value, such as above, the component value may simply be the overall score of each individual or categorical value. For example, if five of the components of the 8PMI component profile are phosphatidylcholine, and three of those components are assigned values of “−2” and two are assigned values of “−1,” the phosphatidylcholine portion of the component profile in this example would be −8, with a normal value being, for example, “0.” In this manner, the component profile value could be a useful single number or score, the actual value or magnitude of which could be an indication of the actual risk of developing an adverse reaction to radiation therapy for cancer, e.g., the “more negative” the value, the greater the risk of developing an adverse reaction to radiation therapy for cancer.

In another embodiment the “component profile value” can be a series of values, numbers, factors, or scores given to the individual components of the overall profile. In another embodiment, the “component profile value” may be a combination of values, numbers, factors, or scores given to individual components of the profile as well as values, numbers, factors, or scores collectively given to a group of components, such as a phosphatidylcholine portion, an acylcarnitine portion, a biogenic amine portion and/or an amino acid portion. In another example, the component profile value may comprise or consist of individual values, number, factors, or scores for specific component as well as values, numbers, factors, or scores for a group on components.

In another embodiment individual values from the components can be used to develop a single score, such as a “combined component index,” which may utilize weighted scores from the individual component values reduced to a diagnostic number value. The combined component index may also be generated using non-weighted scores from the individual component values. When the “combined component index” exceeds a specific threshold level, determined by a range of values developed similarly from control subjects, the individual has a high risk, or higher than normal risk, of developing an adverse reaction to radiation therapy for cancer, whereas maintaining a normal range value of the “combined component index” would indicate a low or minimal risk of developing an adverse reaction to radiation therapy for cancer. In this embodiment, the threshold value would be or could be set by the combined component index from one or more normal subjects.

In another embodiment, the value of the component profile can be the collection of data from the individual measurements and need not be converted to a scoring system, such that the “component profile value” is a collection of the individual measurements of the individual components of the profile.

### Analysis of the Component Profiles

In the methods of the present invention, the component profile may be a metabolite profile, a lipid profile, or a combination thereof.

As used herein, the phrase “metabolite profile” means the combination of a subject's metabolites found in the peripheral blood or portions thereof, such as but not limited to plasma or serum. The metabolite profile is a collection of measurements, such as but not limited to a quantity or level, for individual metabolites taken from a test sample of the subject.

As used herein, the phrase “lipid profile” means the combination of a subject's lipids found in the peripheral blood or portions thereof, such as but not limited to plasma or serum. The lipid profile is a collection of measurements, such as but not limited to a quantity or level, for individual lipid taken from a test sample of the subject.

When assessing if a subject is at risk of developing an adverse reaction after receiving radiation therapy for cancer, for example prostate cancer, the metabolite profile may comprise one or more of the following metabolites: phosphatidylcholine acyl-alkyl C40:1 (PC ae C40:1), phosphatidylcholine acyl-alkyl C40:6 (PC ae C40:6), phosphatidylcholine acyl-alkyl C42:1 (PC ae C42:1), arginine, adenosine, phosphatidylcholine diacyl C26:0 (PC aa C26:0), phosphatidylcholine acyl-alkyl C36:2 (PC ae C36:2), lysophosphatidylcholine acyl C26:1 (LysoPC a C26:1), phosphatidylcholine acyl-alkyl C36:1 (PC ae C36:1), phosphatidylcholine acyl-alkyl C42:0 (PC ac C42:0), sphingomyelin C20:2 (SM C20:2). O-acetyl-1-carnitine, 2-aminoadipic acid, the ratio of chenodeoxycholic acid to deoxycholic acid (CDCA/DCA), lysophosphatidylcholine acyl C20:4 (LysoPC a C20:4), phosphatidylcholine diacyl C34:2 (PC aa C34:2), phosphatidylcholine acyl-alkyl C40:5 (PC ae C40:5), phosphatidylcholine diacyl C36:1 (PC aa C36:1), phosphatidylcholine diacyl C40:5 (PC aa. C40:5), phosphatidylcholine acyl-alkyl C40:3 (PC ae C40:3), lysophosphatidylcholine acyl C18:2 (LysoPC a C18:2), lysophosphatidylcholine acyl C20:3 (LysoPC a C20:3), lysophosphatidylcholine acyl C14:0 (LysoPC a C14:0), geranyl pyrophosphate, glucose4-phosphate, 3-hydroxy-3-methylglutaryl-CoA, geranyl pyrophosphate-0, taurine, 5-aminoimidazole-4-carboxamide ribonucleotide (AICAR), carbamoyl phosphate, uridine triphosphate, fructose 1,6-bisphosphonate, n-acetylomithine, 6-phosphogluconate, tryptophan, xanthurenic acid, palmitric acid, ureidosuccinic acid, alpha-d-glucose, glucosamine 6-phosphate, 1-dihydroorotic acid, carnosine, urea, deoxyinosine, imidazole, 3-hydroxybutanoate, glucose 6-phosphate, dGTP, 5-hydroxytryptophan, 2-deoxyglucose-6-phosphate, 6-phosphogluconate, metanephrine, pantothenate, 4-pyridoxate, methylthioadenosine, phenylacetyl-1-glutamine, ADP, and hydroxyisocaproic acid. Metabolite C species, e.g., C3, denote acylcarnitines (ACs). Phosphocholine (PC) metabolites display combined numbers of carbon atoms for their two acyl groups (sn1 and sn2 positions), e.g., C38, whereas the combined number of double bonds (unsaturation) is displayed after the colon, e.g., C38:6, Acyl group linkages to choline backbone for PCs feature ester (a) or ether (e) linkage, e.g., PC ae C42:1.

In one embodiment, when treating a subject having cancer or determining the risk of cancer recurrence or a late effect after receiving radiation therapy for cancer, the individual levels of each of the metabolites are lower than those compared to normal levels. In another embodiment, the level of one, two, three, four, five, six, seven, eight, nine, ten, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 of the metabolites is lower than the normal level while others, if any, are higher than the normal level. In another embodiment, the individual level of each of the metabolites is higher than those compared to the normal level. In another embodiment, the level of one, two, three, four, five, six, seven, eight, nine, ten, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 of the metabolites is higher than the normal level while others, if any, are higher than the normal level.

The levels of depletion or augmentation of the metabolites compared to normal levels can vary when treating a subject having cancer or determining the risk of an adverse reaction after receiving radiation therapy for cancer, for example prostate cancer. In some embodiments, the levels of any one or more of the metabolites is at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 lower than normal levels. In some embodiments, the levels of any one or more of the metabolites is at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 higher than normal levels. For the purposes of the present invention, the number of “times” the level of a metabolite is lower or higher over normal can be a relative or absolute number of times. In the alternative, the levels of the metabolites may be normalized to a standard and these normalized levels can then be compared to one another to determine if a metabolite is lower or higher.

For the purposes of the present invention, the metabolite profile comprises at least two, three, four, five, six, seven, eight, nine, ten, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or all 50 of the metabolites listed above. If two metabolites are used in generating the metabolite profile, any combination of the two listed above can be used; if three metabolites are used in generating the metabolite profile, any combination of three of the metabolites listed above can be used; if four metabolites are used in generating the metabolite profile, any combination of four of the metabolites listed above can be used; if five metabolites are used in generating the metabolite profile, any combination of five of the metabolites listed above can be used; if six metabolites are used in generating the metabolite profile, any combination of six of the metabolites listed above can be used; if seven metabolites are used in generating the metabolite profile, any combination of seven of the metabolites listed above can be used; and so on. All 50 metabolites can be used in generating the metabolite profile to treat a subject having cancer or to determine risk of an adverse reaction after receiving radiation therapy for cancer.

In embodiments of the invention, the metabolite profile comprises geranyl pyrophosphate, glucose-1-phosphate, and 3-hydroxy-3-methylglutaryl-CoA. In some embodiments, the metabolite profile comprising geranyl pyrophosphate, glucose-1-phosphate, and 3-hydroxy-3-methylglutaryl-CoA may be used to determine whether a subject has an increased risk of tumor recurrence after radiation therapy. In certain embodiments, a subject is determined to have an increased risk of tumor recurrence after radiation therapy, or the subject's level of each metabolite is considered altered as compared to its normal level, when the subject's level of geranyl pyrophosphate, glucose-1-phosphate, and 3-hydroxy-3-methylglutaryl-CoA is each higher than the respective normal level.

In some embodiments, the metabolite profile comprises geranyl pyrophosphate, glucose-1-phosphate, 3-hydroxy-3-methylglutaryl-CoA, and one or more metabolites selected from the following: geranyl pyrophosphate-0, taurine, AICAR, carbamoyl phosphate, uridine tri phosphate, fructose 1,6-bisphosphonate, n-acetylomithine, and 6-phosphogluconate. In some embodiments, the metabolite profile comprising geranyl pyrophosphate, glucose-1-phosphate, 3-hydroxy-3-methylglutaryl-CoA, and one or more metabolites selected from geranyl pyrophosphate-0, taurine, carbamoyl phosphate, uridine triphosphate, fructose bisphosphonate, n-acetylomithine, and 6-phosphogluconate, may be used to determine whether a subject has an increased risk of tumor recurrence after radiation therapy. In certain embodiments, a subject is determined to have an increased risk of tumor recurrence after radiation therapy, or the subject's level of each metabolite is considered altered as compared to its normal level, when the subject's level of geranyl pyrophosphate, glucose-1-phosphate, and 3-hydroxy-3-methylglutaryl-CoA is each higher than the respective normal level; and one or more of the following is determined: the subject's level of AICAR is higher than the normal level, the subject's level of carbamoyl phosphate is higher than the normal level, the subject's level of uridine triphosphate is higher than the normal level, the subject's level of fructose is higher than the normal level, the subject's level of 1,6-bisphosphonate is higher than the normal level, the subject's level of 6-phosphogluconate is higher than the normal level, the subject's level of taurine is lower than the normal level, and the subject's level of n-acetylornithine is lower than the normal level.

In some embodiments, the metabolite profile comprises geranyl pyrophosphate, glucose-1-phosphate, 3-hydroxy-3-methylglutaryl-CoA, and one or more metabolites selected from the following: geranyl pyrophosphate-0, taurine, AICAR, carbamoyl phosphate, uridine triphosphate, fructose 1,6-bisphosphonate, n-acetylomithine, 6-phosphogluconate, tryptophan, xanthurenic acid, palmitric acid, ureidosuccinic acid, alpha-d-glucose, glucosamine 6-phosphate, 1-dihydroorotic acid, carnosine, urea, deoxvinosine, 3-hydroxybutanoate, glucose 6-phosphate, dGGTP, 5-hydroxytryptophan, 2-deoxyglucose-6-phosphate, and 6-phosphogluconate. In some embodiments, the metabolite profile comprising geranyl pyrophosphate, glucose-1-phosphate, 3-hydroxy-3-methylglutaryl-CoA, and one or more metabolites selected from geranyl pyrophosphate-0, taurine, AICAR, carbamoyl phosphate, uridine triphosphate, fructose 1,6-bisphosphonate, n-acetylornithine, 6-phosphogluconate, tryptophan, xanthurenic acid, palmitric acid, ureidosuccinic acid, alpha-d-glucose, glucosamine 6-phosphate, 1-dihydroorotic acid, carnosine, urea, deoxyinosine, imidazole, 3-hydroxybutanoate, glucose 6-phosphate, dGTP, 5-hydroxytryptophan, 2-deoxyglucose-6-phosphate, and 6-phosphogluconate, may be used to determine whether a subject has an increased risk of tumor recurrence after radiation therapy. In certain embodiments, a subject is determined to have an increased risk of tumor recurrence after radiation therapy, or the subject's level of each metabolite is considered altered as compared to its normal level, when the subject's level of geranyl pyrophosphate, glucose-1-phosphate, and 3-hydroxy-3-methylglutaryl-CoA is each higher than the respective normal level; and one or more of the following is determined: the subject's level of AICAR is higher than the normal level, the subject's level of carbamoyl phosphate is higher than the normal level, the subject's level of uridine triphosphate is higher than the normal level, the subject's level of fructose is higher than the normal level, the subject's level of 1,6-bisphosphonate is higher than the normal level, the subject's level of 6-phosphogluconate is higher than the normal level, the subject's level of palmitic acid is higher than the normal level, the subject's level of glucosamine 6-phosphate is higher than the normal level, the subject's level of I-dihydroorotic acid is higher than the normal level, the subject's level of carnosine is higher than the normal level, the subject's level of urea is higher than the normal level, the subject's level of imidazole is higher than the normal level, the subject's level of 3-hydroxybutanoate is higher than the normal level, the subject's level of glucose 6-phosphate is higher than the normal level, the subject's level of 2-deoxyglucose-6-phosphate is higher than the normal level, the subject's level of 6-phosphogluconate is higher than the normal level, the subject's level of taurine is lower than the normal level, the subject's level of n-acetylornithine is lower than the normal level, the subject's level of tryptophan is lower than the normal level, the subject's level of xanthurenic acid is lower than the normal level, the subject's level of ureidosuccinic acid is lower than the normal level, the subject's level of alpha-d-glucose is lower than the normal level, the subject's level of deoxyinosine is lower than the normal level, the subject's level of dGTP is lower than the normal level, and the subject's level of 5-hydroxytryptophan is lower than the normal level.

In embodiments of the invention, the metabolite profile comprises geranyl pyrophosphate, glucose-1-phosphate, and taurine. In some embodiments, the metabolite profile comprising geranyl pyrophosphate, glucose-1-phosphate, and taurine may be used to determine whether a subject has an increased risk of tumor recurrence after radiation therapy. In certain embodiments, a subject is determined to have an increased risk of tumor recurrence after radiation therapy, or the subject's level of each metabolite is considered altered as compared to its normal level, when the subject's level of geranyl pyrophosphate and glucose-1-phosphate is each higher than the respective normal level, and the subject's level of taurine is lower than the normal level.

In some embodiments, the metabolite profile comprises geranyl pyrophosphate, glucose-1-phosphate, taurine, and one or more metabolites selected from the following: geranyl pyrophosphate-0, AICAR, 3-hydroxy-3-methylglutaryl-CoA, carbamoyl phosphate, uridine triphosphate, fructose 1,6-bisphosphonate, n-acetylornithine, and 6-phosphogluconate. In some embodiments, the metabolite profile comprising geranyl pyrophosphate, glucose-1-phosphate, taurine, and one or more metabolites selected from geranyl pyrophosphate-O, AIAR, 3-hydroxy-3-methylglutaryl-CoA, carbamoyl phosphate, uridine triphosphate, fructose 1,6-bisphosphonate, n-acetylornithine, and 6-phosphogluconate, may be used to determine whether a subject has an increased risk of tumor recurrence after radiation therapy. In certain embodiments, a subject is determined to have an increased risk of tumor recurrence after radiation therapy, or the subject's level of each metabolite is considered altered as compared to its normal level, when the subject's level of geranyl pyrophosphate and glucose--phosphate is each higher than the respective normal level and the subject's level of taurine is lower than the normal level; and one or more of the following is determined: the subject's level of AICAR is higher than the normal level, the subject's level of 3-hydroxy-3-methylglutaryl-CoA is higher than the normal level, the subject's level of carbamoyl phosphate is higher than the normal level, the subject's level of uridine triphosphate is higher than the normal level, the subject's level of fructose is higher than the normal level, the subject's level of 1,6-bisphosphonate is higher than the normal level, the subject's level of 6-phosphogluconate is higher than the normal level, the subject's level of taurine is lower than the normal level, and the subject's level of n-acetylomithine is lower than the normal level.

I In some embodiments, the metabolite profile comprises geranyl pyrophosphate, glucose-1-phosphate, taurine, and one or more metabolites selected from the following: geranyl pyrophosphate-0, AICAR, 3-hydroxy-3-methylglutaryl-CoA, carbamoyl phosphate, uridine triphosphate, fructose 1,6-bisphosphonate, n-acetylornithine, 6-phosphogluconate, tryptophan, xanthurenic acid. palmitric acid, ureidosuccinic add, alpha-d-glucose, glucosamine 6-phosphate, 1-dihydroorotic acid, carnosine, urea, deoxyinosine, imidazole, 3-hydroxybutanoate, glucose 6-phosphate, dGTP, 5-hydroxytryptophan, 2-deoxyglucose-6-phosphate, and 6-phosphogluconate. In some embodiments, the metabolite profile comprising geranyl pyrophosphate, glucose-1-phosphate, taurine, and one or more metabolites selected from the following: geranyl pyrophosphate-0, AICAR, 3-hydroxy-3-methylglutaryl-CoA, carbamoyl phosphate, uridine triphosphate, fructose 1,6-bisphosphonate, n-acetylornithine, 6-phosphogluconate, tryptophan, xanthurenic acid, palmitric acid, ureidosuccinic acid, alpha-d-glucose, glucosamine 6-phosphate, 1-dihydroorotic acid, carnosine, urea, deoxyinosine, imidazole, 3-hydroxybutanoate, glucose 6-phosphate, dGTP, 5-hydroxytryptophan, 2-deoxyglucose-6-phosphate, and 6-phosphogluconate, may be used to determine whether a subject has an increased risk of tumor recurrence after radiation therapy. In certain embodiments, a subject is determined to have an increased risk of tumor recurrence after radiation therapy, or the subject's level of each metabolite is considered altered as compared to its normal level, when the subject's level of geranyl pyrophosphate and glucose-1-phosphate is each higher than the respective normal level and the subject's level of taurine is lower than the normal level; and one or more of the following is determined: the subject's level of ATCAR is higher than the normal level, the subject's level of 3-hydroxy-3-methylglutaryl-CoA is higher than the normal level, the subject's level of carbamoyl phosphate is higher than the normal level, the subject's level of uridine triphosphate is higher than the normal level, the subject's level of fructose is higher than the normal level, the subject's level of 1,6-bisphosphonate is higher than the normal level, the subject's level of 6-phosphogluconate is higher than the normal level, the subject's level of palmitic acid is higher than the normal level, the subject's level of glucosamine 6-phosphate is higher than the normal level, the subject's level of ]-dihydroorotic acid is higher than the normal level, the subject's level of carnosine is higher than the normal level, the subject's level of urea is higher than the normal level, the subject's level of imidazole is higher than the normal level, the subject's level of 3-hydroxybutanoate is higher than the normal level, the subject's level of glucose 6-phosphate is higher than the normal level, the subject's level of 2-deoxyglucose-6-phosphate is higher than the normal level, the subject's level of 6-phosphogluconate is higher than the normal level, the subject's level of taurine is lower than the normal level, the subject's level of n-acetylomithine is lower than the normal level, the subject's level of tryptophan is lower than the normal level, the subject's level of xanthurenic acid is lower than the normal level, the subject's level of ureidosuccinic acid is lower than the normal level, the subject's level of alpha-d-glucose is lower than the normal level, the subject's level of deoxyinosine is lower than the normal level, the subject's level of dGTP is lower than the normal level, and the subject's level of 5-hydroxytryptophan is lower than the normal level.

In embodiments of the invention, the metabolite profile comprises PC ae C40:1, PC ae C40:6, PC ae arginine, adenosine, PC aa C26:0, PC ae C36:2. and LysoPC a C26:1. In some embodiments, the metabolite profile comprising PC ae C40:1, PC ae C40:6, PC ae C42:1, arginine, adenosine, PC aa C26:0, PC ae C36:2, and LysoPC a C26:1 may be used to determine whether a subject has an increased risk of tumor recurrence after radiation therapy. In certain embodiments, a subject is determined to have an increased risk of tumor recurrence after radiation therapy, or the subject's level of each metabolite is considered altered as compared to its normal level, when the subject's level of PC ae C40:1, PC ae C40:6, PC ae C42:1, adenosine, PC aa C26:0, and LysoPC a C26:1 is each lower than the respective normal level, and the subject's level of arginine and PC ae C36:2 is each higher than the respective normal level.

In embodiments of the invention, the metabolite profile comprises metanephrine, tryptophan, xanthurenic acid, and pantothenate. In some embodiments, the metabolite profile comprising metanephrine, tryptophan, xanthurenic acid, and pantothenate may be used to determine whether a subject has an increased risk of one or more late effects, such as rectal toxicity or urinary toxicity, after radiation therapy. In certain embodiments, a subject is determined to have an increased risk of late effects after radiation therapy, or the subject's level of each metabolite is considered altered as compared to its normal level, when the subject's level of metanephrine, tryptophan, xanthurenic acid, and pantothenate is each lower than the respective normal level.

In some embodiments, the metabolite profile comprises metanephrine, tryptophan, xanthurenic acid, and pantothenate, and one or more metabolites selected from the following: 4-pyridoxate, methylthioadenosine, tryptophan, phenylacetyl-l-glutamine, ADP, n-acetylornithine, and hydroxyisocaproic acid. In some embodiments, the metabolite profile comprising metanephrine, tryptophan, xanthurenic acid, and pantothenate, and one or more metabolites selected from 4-pyridoxate, methylthioadenosine, tryptophan, phenylacetyl-l-glutamine, ADP, n-acetylornithine, and hydroxyisocaproic acid, may be used to determine whether a subject has an increased risk of one or more late effects, such as rectal toxicity or urinary toxicity, after radiation therapy. In certain embodiments, a subject is determined to have an increased risk of late effects after radiation therapy, or the subject's level of each metabolite is considered altered as compared to its normal level, when the subject's level of metanephrine, tryptophan, xanthurenic acid, and pantothenate is each lower than the respective normal level, and one or more of the following is determined: the subject's level of 4-pyridoxate is lower than the normal level, the subject's level of methylthioadenosine is lower than the normal level, the subject's level of tryptophan is lower than the normal level, the subject's level of phenylacetyl-l-glutamine is lower than the normal level, the subject's level of ADP is lower than the normal level, the subject's level of n-acetylornithine is lower than the normal level, and the subject's level of hydroxyisocaproic acid is lower than the normal level.

In embodiments of the invention, the metabolite profile comprises PC ae C36:1, PC ae C42:0, SM C20:2, O-acetyl-1-carnitine, 2-aminoadipic acid, and the ratio of CDCA/DCA. In some embodiments, the metabolite profile comprising PC ae C36:1, PC ae C42:0, SM C20:2, O-acetyl-1-carnitine, 2-aminoadipic acid, and the ratio of CDCA/DCA may be used to determine whether a subject has an increased risk of one or more late effects, such as rectal toxicity, after radiation therapy. In certain embodiments, a subject is determined to have an increased risk of late effects after radiation therapy, or the subject's level of each metabolite is considered altered as compared to its normal level, when the subject's level of PC ae C42:0, SM C20:2, 0-acetyl-1-carnitine, and 2-aminoadipic acid is each higher than the respective normal level, the subject's level of PC ae C36:1 is lower than the normal level, and the ratio of the subject's levels of CDCA/DCA is lower than the ratio of normal of CDCA/DCA.

In embodiments of the invention, the metabolite profile comprises LysoPC a C20:4, PC aa C34:2. PC ae C40:5, PC aa. C36:1, PC aa C40:5, PC ae C40:3. LysoPC a C18:2, LysoPC a C20:3, and LysoPC a C14:0, In some embodiments, the metabolite profile comprising LysoPC a C20:4, PC aa C34:2, PC ae C40:5, PC aa C36:1, PC aa C40:5, PC ae C40:3, LysoPC a C18:2, LysoPC a C20:3, and LysoPC a C14:0 may be used to determine whether a subject has an increased risk of one or more late effects, such as urinary toxicity, after radiation therapy. In certain embodiments, a subject is determined to have an increased risk of late effects after radiation therapy, or the subject's level of each metabolite is considered altered as compared to its normal level, when the subject's level of LysoPC a C20:4, PC aa C34:2, LysoPC a C18:2, LysoPC a C20:3, and LysoPC a C14:0 is each higher than the respective normal level; and the subject's level of PC ae C40:5, PC aa C36:1, PC aa C40:5, and PC ae C40:3 is each lower than the respective normal level.

Tables 1-3 and 7-18 in the Examples list exemplary analysis of the metabolites used for each specific adverse reaction to receiving radiation therapy for cancer, for example prostate cancer. Herein, a “mean fold change” of one (1) indicates no change while values less than one indicate a negative change in the diagnostic group as compared to the normal control (NC). Herein, values greater than one indicate a positive change in the diagnostic group compared to NC.

When assessing if a subject is at risk of developing an adverse reaction after receiving radiation therapy for cancer, the lipid profile may comprise one or more of the following lipids: LPA 18:0, LPA 18:1, LPA 16:0, LPA 18:2, LPC 20:2, CER 24:0, LPC 20:3, LPC 20:0, LPI 16:1, LPA 16:1, LPC 22:0, LPC 20:1, PS 18:0/18:1, lysophosphatidylethanolamine (LPE) 22:0, LPC 22:5, phosphatidylglycerol (PG) 18:0/20:4, phosphatidylserine (PS) 16:1/18:2:2, PS 16:1/18:1:2, PG 18:0/20:4:2, LPC 17:0, CE 16:0, LPC 24:1, PS 16:0/18:2, LPE 16:1, LPC 24:0, CER 14:0, PG 16:0/18:0:2, PS 18:1/18:2:2, LPE 24:0, PS 16:0/16:1:2, PS 18:0/18:1:2, PG 16:0/18:0, PG 16:0/18:1, PG 16:0/18:1:2, PS 18:0/18:2, LPC 20:4, LPC 16:1, PC 16:0/16:0, PS 18:0/18:0, PS 16:1/18:0:2, LPC 22:6, PG 18:0/18:1, cholesteryl ester (CE) 22:4, PG 18:0/18:1, phosphatidic acid (PA) 18:0/20:3, PG 18:0/18:2, LPC 15:0, PC 14:0/18:2, CE 18:1, PA 18:1/18:2, PG 16:0/20:3, PG 18:0/18:2, CE 17:0, PG 18:0/18:0, PG 16:0/20:2 , PS 16:0/20:3, FFA 18:2, free fatty acid (FFA) 18:0, PG 18:1/18:1, CE 18:0, PG 16:1/18:1, PS 16:0/18:0, PA 18:0/18:2, PG 18:1/18:2 , PS 16:0/18:1, LPC 18:1, PG 16:0/18:2, PEP-18:1/20:4, hexosylceramide (HCER) 18:0, cholesterol, CE 18:3, LPC 14:0, PG 16:0/18:2, PG 16:0/20:2, PG 16:0/20:3, DAG 16:0/18:0, DAG 18:1/18:1, DAG 16:1/18:2, PG 16:0/16:0, PE 18:2/20:2, PG 18:1/18:2, LPE 18:3, LPC 22:1, PEP-18:0/20:4, CE 18:2, PG 18:2/18:2, PS 16:1/18:0, HCER 20:0, FFA 18:1, PG 16:1/18:0:2, LPC 18:2, FFA 16:0, PS 16:0/18:0:2, CE 20:4, PC 16:0/14:0, CE 22:5, LCER 22:0, PC 14:0/16:1, CER 22:0, PG 16:1/18:0, CER 24:1, PC 14:0/18:1, sphingomyelin (SM) d18:1/12:0, PE P-18:1/22:6, FFA 22:2, PE 16:0/16:0, PI 14:0/18:1, LPC 18:3, PEP-18:0/22:6, LPC 18:0, FFA 12:0, PC 12:0/18:2, PG 16:1/18:2:2, FFA 20:4, PG 16:1/18:1:2, CE 22:6, PC 18:0/14:0, FFA 17:0, PC 14:0/18:3, CE 20:3, SM d18:1/14:1, PE P-18:2/20:4, PE P-18:1/22:5, FFA 14:0, PE 18:0/15:0, HCER 20:1, PC 14:0/14:0, triacylglycerol (TAG) 52:2/FA16:0, PE P-16:0/22:6, LPC 20:5, PG 16:0/16:1, FFA 18:4, PE P-18:2/22:6, FFA 20:0, PE P-16:0/16:1, FFA 15:0, PC 18:0/18:0, and HCER 14:0.

In one embodiment, when treating a subject having cancer or determining the risk of cancer recurrence or a late effect after receiving radiation therapy for cancer, the individual levels of each of the lipids are lower than those compared to normal levels. In another embodiment, level of one, two, three, four, five, six, seven, eight, nine, ten, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, or 136 of the lipids is lower than the normal level while others, if any, are higher than the normal level. In another embodiment, the individual level of each of the lipids is higher than those compared to the normal level. In another embodiment, level of one, two, three, four, five, six, seven, eight, nine, ten, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, or 136 of the lipids is higher than normal level while others, if any, are higher than normal level.

The levels of depletion or augmentation of the lipids compared to normal levels can vary when treating a subject having cancer or determining the risk of an adverse reaction after receiving radiation therapy for cancer, for example prostate cancer. In some embodiments, the levels of any one or more of the lipids is at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 lower than normal levels. In some embodiments, the levels of any one or more of the lipids is at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 higher than normal levels. For the purposes of the present invention, the number of “times” the level of a lipid is lower or higher over normal can be a relative or absolute number of times. In the alternative, the levels of the lipids may be normalized to a standard and these normalized levels can then be compared to one another to determine if a lipid is lower or higher.

For the purposes of the present invention, the lipid profile comprises at least two, three, four, five, six, seven, eight, nine, ten, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, or 136 of the lipids listed above. If two lipids are used in generating the lipid profile, any combination of the two listed above can be used; if three lipids are used in generating the lipid profile, any combination of three of the lipids listed above can be used; if four lipids are used in generating the lipid profile, any combination of four of the lipids listed above can be used; if five lipids are used in generating the lipids profile, any combination of five of the lipids listed above can be used; if six lipids are used in generating the lipid profile, any combination of six of the lipids listed above can be used; if seven lipids are used in generating the metabolite profile, any combination of seven of the lipids listed above can be used; and so on. All 136 lipids can be used in generating the lipid profile to treat a subject having cancer or to determine risk of an adverse reaction after receiving radiation therapy for cancer.

In embodiments of the invention, the lipid profile comprises LPA 18:0, LPA 16:0, LPC 20:2, CER 24:0, and LPI 16:1. In some embodiments, the lipid profile comprising LPA 18:0, LPA 16:0, LPC 20:2, CER 24:0, and LPI 16:1 may be used to determine whether a subject has an increased risk of tumor recurrence after radiation therapy. In certain embodiments, a subject is determined to have an increased risk of tumor recurrence after radiation therapy, or the subject's level of each lipid is considered altered as compared to its normal level, when the subject's level of LPA 18:0, LPA 16:0, and LPC 20:2 is each higher than the respective normal level, and the subject's level of CER 24:0 and LPI 16:1 is each lower than the respective normal level.

In some embodiments, the lipid profile comprises LPA 18:0, LPA 16:0, LPC 20:2, CER 24:0, and LPI 16:1, and one or more lipids selected from the following: LPA 18:1, LPA 18:2, LPC 20:3, LPC 20:0, LPA 16:1, LPC 22:0, LPC 20:1, PS 18:0/18:1, LPE 22:0, LPC 22:5, PG 18:0/20:4, PS 16:1/18:2:2, PS 16:1/18:1:2, PG 18:0/20:4:2, LPC 17:0, CE 16:0, LPC 24:1, PS 16:0/18:2, LPE 16:1, LPC 24:0, CER 14:0, PG 16:0/18:0:2, PS 18:1/18:2:2, LPE 24:0, PS 16:0/16:1:2, PS 18:0/18:1:2, PG 16:0/18:0, PG 16:0/18:1, PG 16:0/18:1:2, PS 18:0/18:2, LPC 20:4, LPC 16:1, PC 16:0/16:0, PS 18:0/18:0, PS 16:1/18:0:2, LPC 22:6, PG 18:0/18:1, CE 22:4, PG 18:0/18:1, PA 18:0/20:3, PG 18:0/18:2, LPC 15:0, PC 14:0/18:2, CE 18:1, PA 18:1/18:2, PG 16:0/20:3, PG 18:0/18:2, CE 17:0, PG 18:0/18:0, PG 16:0/20:2 , PS 16:0/20:3, FFA 18:2, FFA 18:0, PG 18:1/18:1, CE 18:0, PG 16:1/18:1, PS 16:0/18:0, PA 18:0/18:2, PG 18:1/18:2 , PS 16:0/18:1, LPC 18:1, PG 16:0/18:2, PE P-18:1/20:4, HCER 18:0, Cholesterol, CE 18:3, LPC 14:0, PG 16:0/18:2, PG 16:0/20:2, PG 16:0/20:3, DAG 16:1/18:2, PG 16:0/16:0, PE 18:2/20:2, PG 18:1/18:2, LPE 18:3, LPC 22:1, PE P-18:0/20:4, CE 18:2, PG 18:2/18:2, PS 16:1/18:0, HCER 20:0, FFA 18:1, PG 16:1/18:0:2, LPC 18:2, FFA 16:0, PS 16:0/18:0:2, CE 20:4, PC 16:0/14:0, CE 22:5, LCER 22:0, PC 14:0/16:1, CER 22:0, PG 16:1/18:0, CER 24:1, PC 14:0/18:1, SM d18:1/12:0, PE P-18:1/22:6, FFA 22:2, PE 16:0/16:0, PI 14:0/18:1, LPC 18:3, PE P-18:0/22:6, LPC 18:0, FFA 12:0, PC 12:0/18:2, PG 16:1/18:2:2, FFA 20:4, PG 16:1/18:1:2, CE 22:6, PC 18:0/14:0, FFA 17:0, PC 14:0/18:3, CE 20:3, SM d18:1/14:1, PE P-18:2/20:4, PE P-18:1/22:5, FFA 14:0, PE 18:0/15:0, HCER 20:1, PC 14:0/14:0, TAG 52:2/FA16:0, PE P-16:0/22:6, LPC 20:5, PG 16:0/16:1, FFA 18:4, PE P-18:2/22:6, FFA 20:0, PE P-16:0/16:1, FFA 15:0, PC 18:0/18:0, and HCER 14:0. in some embodiments, the lipid profile comprising LPA 18:0, LPA 16:0, LPC 20:2, CER 24:0, and LPI 16:1, and one or more lipids selected from LPA 18:1, LPA 18:2, LPC 20:3, LPC 20:0, LPA 16:1, LPC 22:0, LPC 20:1, PS 18:0/18:1, LPE 22:0, LPC 22:5, PG 18:0/20:4, PS 16:1/18:2:2, PS 16:1/18:1:2, PG 18:0/20:4:2, LPC 17:0, CE 16:0, LPC 24:1, PS 16:0/18:2, LPE 16:1, LPC 24:0, CER 14:0, PG 16:0/18:0:2, PS 18:1/18:2:2, LPE 24:0, PS 16:0/16:1:2, PS 18:0/18:1:2, PG 16:0/18:0, PG 16:0/18:1, PG 16:0/18:1:2, PS 18:0/18:2, LPC 20:4, LPC 16:1, PC 16:0/16:0, PS 18:0/18:0, PS 16:1/18:0:2, LPC 22:6, PG 18:0/18:1, CE 22:4, PG 18:0/18:1, PA 18:0/20:3, PG 18:0/18:2, LPC 15:0, PC 14:0/18:2, CE 18:1, PA 18:1/18:2, PG 16:0/20:3, PG 18:0/18:2, CE 17:0, PG 18:0/18:0, PG 16:0/20:2 , PS 16:0/20:3, FFA 18:2, FFA 18:0, PG 18:1/18:1, CE 18:0, PG 16:1/18:1, PS 16:0/18:0, PA 18:0/18:2, PG 18:1/18:2 , PS 16:0/18:1, LPC 18:1, PG 16:0/18:2, PE P-18:1/20:4, HCER 18:0, Cholesterol, CE 18:3, LPC 14:0, PG 16:0/18:2, PG 16:0/20:2, PG 16:0/20:3, DAG 16:1/18:2, PG 16:0/16:0, PE 18:2/20:2, PG 18:1/18:2, LPE 18:3, LPC 22:1, PE P-18:0/20:4, CE 18:2, PG 18:2/18:2, PS 16:1/18:0, HCER 20:0, FFA 18:1, PG 16:1/18:0:2, LPC 18:2, FFA 16:0, PS 16:0/18:0:2, CE 20:4, PC 16:0/14:0, CE 22:5, LCER 22:0, PC 14:0/16:1, CER 22:0, PG 16:1/18:0, CER 24:1, PC 14:0/18:1, SM d18:1/12:0, PEP-18:1/22:6, FFA 22:2, PE 16:0/16:0, PI 14:0/18:1, LPC 18:3, PE P-18:0/22:6, LPC 18:0, FFA 12:0, PC 12:0/18:2, PG 16:1/18:2:2, FFA 20:4, PG 16:1/18:1:2, CE 22:6, PC 18:0/14:0, FFA 17:0, PC 14:0/18:3, CE 20:3, SM d18:1/14:1, PE P-18:2/20:4, PE P-18:1/22:5, FFA 14:0, PE 18:0/15:0, HCER 20:1, PC 14:0/14:0, TAG 52:2/FA16:0, PE P-16:0/22:6, LPC 20:5, PG 16:0/16:1, FFA 18:4, PE P-18:2/22:6, FFA 20:0, PE P-16:0/16:1, FFA 15:0, PC 18:0/18:0, and HCER 14:0, may be used to determine whether a subject has an increased risk of tumor recurrence after radiation therapy. In certain embodiments, a subject is determined to have an increased risk of tumor recurrence after radiation therapy, or the subject's level of each lipid is considered altered as compared to its normal level, when the subject's level of LPA 18:0, LPA 16:0, and LPC 20:2 is each higher than the respective normal level, the subject's level of CER 24:0 and LPI 16:1 is each lower than the respective normal level; and one or more of the following is determined: the subject's level of LPA 18:1 is higher than the normal level, the subject's level of LPA 18:2 is higher than the normal level, the subject's level of LPC 20:3 is higher than the normal level, the subject's level of LPC 20:0 is higher than the normal level, the subject's level of LPC 22:0 is higher than the normal level, the subject's level of LPC 20:1 is higher than the normal level, the subject's level of LPE 22:0 is higher than the normal level, the subject's level of LPC 22:5 is higher than the normal level, the subject's level of LPC 17:0 is higher than the normal level, the subject's level of LPC 24:1 is higher than the normal level, the subject's level of LPE 16:1 is higher than the normal level, the subject's level of LPC 24:0 is higher than the normal level, the subject's level of LPE 24:0 is higher than the normal level, the subject's level of LPC 20:4 is higher than the normal level, the subject's level of LPC 16:1 is higher than the normal level, the subject's level of PC 16:0/16:0 is higher than the normal level, the subject's level of LPC, 22:6 is higher than the normal level, the subject's level of LPC 15:0 is higher than the normal level, the subject's level of PC 14:0/18:2 is higher than the normal level, the subject's level of LPC 18:1 is higher than the normal level, the subject's level of HCER 18:0 is higher than the normal level, the subject's level of LPC 14:0 is higher than the normal level, the subject's level of DAG 16:1/18:2 is higher than the normal level, the subject's level of LPE 18:3 is higher than the normal level, the subject's level of LPC 22:1 is higher than the normal level, the subject's level of HCER 20:0 is higher than the normal level, the subject's level of LPC 18:2 is higher than the normal level, the subject's level of PC 16:0/14:0 is higher than the normal level, the subject's level of PC 14:0/16:1 is higher than the normal level, the subject's level of PC 14:0/18:1 is higher than the normal level, the subject's level of SM d18:1/12:0 is higher than the normal level, the subject's level of PE 16:0/16:0 is higher than the normal level, the subject's level of ITC 18:3 is higher than the normal level, the subject's level of LPC 18:0 is higher than the normal level, the subject's level of PC 12:0/18:2 is higher than the normal level, the subject's level of PC 18:0/14:0 is higher than the normal level, the subject's level of PC 14:0/18:3 is higher than the normal level, the subject's level of SM d18:1/14:1 is higher than the normal level, the subject's level of PE 18:0/15:0 is higher than the normal level, the subject's level of HCER 20:1 is higher than the normal level, the subject's level of PC 14:0/14:0 is higher than the normal level, the subject's level of LPC 20:5 is higher than the normal level, the subject's level of PE P-16:0/16:1 is higher than the normal level, the subject's level of PC 18:0/18:0 is higher than the normal level, the subject's level of HCER 14:0 is higher than the normal level, the subject's level of LPA 16:1 is lower than the normal level, the subject's level of PS 18:0,18:1 is lower than the normal level. the subject's level of PG 18:0/20:4 is lower than the normal level, the subject's level of PS 16:1/18:2:2 is lower than the normal level, the subject's level of PS 16:1/18:1:2 is lower than the normal level, the subject's level of PG 18:0/20:4:2 is lower than the normal level, the subject's level of CE 16:0 is lower than the normal level, PS 16:0/18:2 is lower than the normal level, the subject's level of CER 14:0 is lower than the normal level, the subject's level of PG 16:0/18:0:2 is lower than the normal level, the subject's level of PS 18:1/18:2:2 is lower than the normal level, the subject's level of PS 16:0/16:1:2 is lower than the normal level, the subject's level of PS 18:0/18:1:2 is lower than the normal level, the subject's level of PG 16:0/18:0 is lower than the normal level, the subject's level of PG 16:0/18:1 is lower than the normal level, the subject's level of PG 16:0/18:1:2 is lower than the normal level, the subject's level of PS 18:0/18:2 is lower than the normal level, the subject's level of PS 18:0/18:0 is lower than the normal level, the subject's level of PS 16:1/18:0:2 is lower than the normal level, the subject's level of PG 18:0/18:1 is lower than the normal level, the subject's level of CE 22:4 is lower than the normal level, the subject's level of PG 18:0/18:1 is lower than the normal level, the subject's level of PA 18:0/20:3 is lower than the normal level, the subject's level of PG 18:0/18:2 is lower than the normal level, the subject's level of CE 18:1 is lower than the normal level, the subject's level of PA 18:1/18:2 is lower than the normal level, the subject's level of PG 16:0/20:3 is lower than the normal level, the subject's level of PG 18:0/18:2 is lower than the normal level, the subject's level of CE 17:0 is lower than the normal level, the subject's level of PG 18:0/18:0 is lower than the normal level, the subject's level of PG 16:0/20:2 is lower than the normal level, the subject's level of PS 16:0/20:3 is lower than the normal level, the subject's level of FFA 18:2 is lower than the normal level, the subject's level of FFA 18:0 is lower than the normal level, the subject's level of PG 18:1/18:1 is lower than the normal level, the subject's level of CE 18:0 is lower than the normal level, the subject's level of PG 16:1/18:1 is lower than the normal level, the subject's level of PS 16:0/18:01s lower than the normal level, the subject's level of PA 18:0/18:2 is lower than the normal level, the subject's level of PG 18:1/18:2 is lower than the normal level, the subject's level of PS 16:0/18:1 is lower than the normal level, the subject's level of PG 16:0/18:2 is lower than the normal level, the subject's level of PE P-18:1/20:4 is lower than the normal level, the subject's level of cholesterol is lower than the normal level, the subject's level of CE 18:3 is lower than the normal level, the subject's level of PG 16:0/18:2 is lower than the normal level, the subject's level of PG 16:0/20:2 is lower than the normal level, the subject's level of PG 16:0/20:3 is lower than the normal level, the subject's level of PG 16:0/16:0 is lower than the normal level, the subject's level of PE 18:2/20:2 is lower than the normal level, the subject's level of PG 18:1/18:2 is lower than the normal level, the subject's level of PE P-18:0/20:4 is lower than the normal level, the subject's level of CE 18:2 is lower than the normal level, the subject's level of PG 18:2/18:2 is lower than the normal level, the subject's level of :PS 16:1/18:0 is lower than the normal level, the subject's level of FFA 18:1 is lower than the normal level, the subject's level of PG 16:1/18:0:2 is lower than the normal level, the subject's level of FFA 16:0 is lower than the normal level, the subject's level of PS 16:0/18:0:2 is lower than the normal level, the subject's level of CE 20:4 is lower than the normal level, the subject's level of CE 22:5 is lower than the normal level, the subject's level of LCER 22:0 is lower than the normal level, the subject's level of CER 22:0 is lower than the normal level, the subject's level of PG 16:1/18:0 is lower than the normal level, the subject's level of CER 24:1 is lower than the normal level, the subject's level of PE P-18:1/22:6 is lower than the normal level, the subject's level of FFA 22:2 is lower than the normal level, the subject's level of PI 14:0/18:1 is lower than the normal level, the subject's level of PE P-18:0/22:6 is lower than the normal level, the subject's level of FFA 12:0 is lower than the normal level, the subject's level of PG 16:1/18:2:2 is lower than the normal level, the subject's level of FFA 20:4 is lower than the normal level, the subject's level of PG 16:1/18:1:2 is lower than the normal level, the subject's level of CE 22:6 is lower than the normal level, the subject's level of FFA 17:0 is lower than the normal level, the subject's level of CE 20:3 is lower than the normal level, the subject's level of PE P-18:2/20:4 is lower than the normal level, the subject's level of PE P-18:1/22:5 is lower than the normal level, the subject's level of FFA 14:0 is lower than the normal level. the subject's level of TAG 52:2/FA16:0 is lower than the normal level. the subject's level of PE Pl 6:0/22:6 is lower than the normal level, the subject's level of PG 16:0/16:1 is lower than the normal level, the subject's level of FFA 18:4 is lower than the normal level, the subject's level of PE P-18:2/22:6 is lower than the normal level, the subject's level of FFA 20:0 is lower than the normal level, and the subject's level of FFA 15:0 is lower than the normal level.

In embodiments of the invention, the lipid profile comprises LPA 18:0, DAG 16:0/18:0, LPA 16:0, and DAG 18:1/18:1. In some embodiments, the lipid profile comprising LPA 18:0, DAG 16:0/18:0, LPA 16:0, and DAG 18:1/18:1 may be used to determine whether a subject has an increased risk of one or more late effects, such as rectal toxicity or urinary toxicity, after radiation therapy. In certain embodiments, a subject is determined to have an increased risk of late effects after radiation therapy, or the subject's level of each lipid is considered altered as compared to its normal level, when the subject's level of LPA18:0 and LPA 16:0 is each higher than the respective normal level, and the subject's level of DAG 16:0/18:1 and DAG 18:1/18:1 is each lower than the respective normal level.

In embodiments of the invention, the lipid profile comprises LPA 18:0, DAG 16:0/18:0, LPA 16:0, and DAG 18:1/18:1, and one or more lipids selected from the following: LPI 16:0, LPA 18:2, LPA 18:1, PI 16:1-18:1:2, CE 201, LPA 16:1, FFA 202, PC 180/225, and PE 180:225. In some embodiments, the lipid profile comprising LPA 18:0, DAG 16:0/18:0, LPA 16:0, and DAG 18:1/18:1, and one or more of LPI 16:0, LPA 18:2, LPA 18:1, PI 16:1-18:1:2, CE 201, LPA 16:1, FFA 202, PC 180/225, and PE 180:225, may be used to determine whether a subject has an increased risk of one or more late effects, such as rectal toxicity or urinary toxicity, after radiation therapy. In certain embodiments, a subject is determined to have an increased risk of late effects after radiation therapy, or the subject's level of each lipid is considered altered as compared to its normal level, when the subject's level of LPA18:0 and LPA 16:0 is each higher than the respective normal level, and the subject's level of DAG 16:0/18:1 and DAG 18:1/18:1 is each lower than the respective normal level; and one or more of the following is determined: the subject's level of LPI 16:0 is lower than the normal level, the subject's level of LPA 18:2 is higher than the normal level, the subject's level of LPA 18:1 is higher than the normal level, the subject's level of PI 16:1-18:1:2 is lower than the normal level, the subject's level of CE 201 is higher than the normal level, the subject's level of LPA 16:1 is higher than the normal level, the subject's level of FFA 202 is higher than the normal level, the subject's level of PC 180/225 is lower than the normal level, and the subject's level of PE 180:225 is lower than the normal level.

When assessing if a subject is at risk of developing an adverse reaction after receiving radiation therapy for cancer, the component profile may comprise one or more of metabolites and lipids listed herein. In embodiments of the invention, the component profile comprises geranyl pyrophosphate, glucose-1-phosphate, 3-hydroxy-3-methylglutaryl-CoA, LPA 18:0, LPA 16:0, LPC 20:2, CER 24:0, and LPI 16:1. In some embodiments, the component profile comprising geranyl pyrophosphate, glucose-1-phosphate, 3-hydroxy-3-methylglutaryl-CoA, LPA 18:0, LPA 16:0, LPC 20:2, CER 24:0, and LPI 16:1, may be used to determine whether a subject has an increased risk of tumor recurrence after radiation therapy. In certain embodiments, a subject is determined to have an increased risk of tumor recurrence after radiation therapy, or the subject's level of each metabolite and lipid is considered altered as compared to its normal level, when the subject's level of geranyl pyrophosphate, glucose-1-phosphate, 3-hydroxy-3-methylglutaryl-CoA, LPA 18:0, LPA 16:0, and LPC 20:2 is each higher than the respective normal level, and the subject's level of CER 24:0 and LPI 16:1 is each lower than the respective normal level.

In embodiments of the invention, the component profile comprises geranyl pyrophosphate, glucose-1-phosphate, 3-hydroxy-3-methylglutaryl-CoA, LPA 18:0, LPA 16:0, LPC 20:2, CER 24:0, and LPI 16:1, and one or more of the following: geranyl pyrophosphate-0, taurine. AICAR, carbarnoyl phosphate, uridine triphosphate, fructose 1,6-bisphosphonate, n-acetylornithine, 6-phosphogluconate, tryptophan, xanthurenic acid, palmitric acid, ureidosuccinic acid, alpha-d-glucose, glucosamine 6-phosphate, 1-dihydroorotic add, carnosine, urea, deoxyinosine, imidazole, 3-hydroxybutanoate, glucose 6-phosphate, dGTP, 5-hydroxytryptophan, 2-deoxyglucose-6-phosphate, 6-phosphogluconate, LPA 18:1, LPA 18:2, LPC 20:3, LPC 20:0, LPA 16:1, LPC 22:0, LPC 20:1, PS 18:0/18:1, LPE 22:0, LPC 22:5, PG 18:0/20:4, PS 16:1/18:2:2, PS 16:1/18:1:2, PG 18:0/20:4:2, LPC 17:0, CE 16:0, LPC 24:1, PS 16:0/18:2, LPE 16:1, LPC 24:0, CER 14:0, PG 16:0/18:0:2, PS 18:1/18:2:2, LPE 24:0, PS 16:0/16:1:2, PS 18:0/18:1:2, PG 16:0/18:0, PG 16:0/18:1, PG 16:0/18:1:2, PS 18:0/18:2, LPC 20:4, LPC 16:1, PC 16:0/16:0, PS 18:0/18:0, PS 16:1/18:0:2, LPC 22:6, PG 18:0/18:1, CE 22:4, PG 18:0/18:1, PA 18:0/20:3, PG 18:0/18:2, LPC 15:0, PC 14:0/18:2, CE 18:1, PA 18:1/18:2, PG 16:0/20:3, PG 18:0/18:2, CE 17:0, PG 18:0/18:0, PG 16:0/20:2 , PS 16:0/20:3, FFA 18:2, FFA 18:0, PG 18:1/18:1, CE 18:0, PG 16:1/18:1, PS 16:0/18:0, PA 18:0/18:2, PG 18:1/18:2 , PS 16:0/18:1, LPC 18:1, PG 16:0/18:2, PE P-18:1/20:4, HCER 18:0, Cholesterol, CE 18:3, LPC 14:0, PG 16:0/18:2, PG 16:0/20:2, PG 16:0/20:3, DAG 16:1/18:2, PG 16:0/16:0, PE 18:2/20:2, PG 18:1/18:2, LPE 18:3, LPC 22:1, PE P-18:0/20:4, CE 18:2, PG 18:2/18:2, PS 16:1/18:0, HCER 20:0, FFA 18:1, PG 16:1/18:0:2, LPC 18:2, FFA 16:0, PS 16:0/18:0:2, CE 20:4, PC 16:0/14:0, CE 22:5, LCER 22:0, PC 14:0/16:1, CER 22:0, PG 16:1/18:0, CER 24:1, PC 14:0/18:1, SM d18:1/12:0, PE P-18:1/22:6, FFA 22:2, PE 16:0/16:0, PI 14:0/18:1, LPC 18:3, PE P-18:0/22:6, LPC 18:0, FFA 12:0, PC 12:0/18:2, PG 16:1/18:2:2, FFA 20:4, PG 16:1/18:1:2, CE 22:6, PC 18:0/14:0, FFA 17:0, PC 14:0/18:3, CE 20:3, SM d18:1/14:1, PE P-18:2/20:4, PE P-18:1/22:5, FFA 14:0, PE 18:0/15:0, HCER 20:1, PC 14:0/14:0, TAG 52:2/FA16:0, PE P-16:0/22:6, LPC 20:5, PG 16:0/16:1, FFA 18:4, PE P-18:2/22:6, FFA 20:0, PE P-16:0/16:1, FFA 15:0, PC 18:0/18:0, and HCER 14:0. In some embodiments, the component profile comprising geranyl pyrophosphate, glucose-1-phosphate, 3-hydroxy-3-methylglutaryl-CoA, LPA 18:0, LPA 16:0, LPC 20:2, CER 24:0, and LPI 16:1, and one or more of geranyl pyrophosphate-O, taurine, AICAR, carbamoyl phosphate, uridine triphosphate, fructose I,6-bisphosphonate, n-acetylornithine, 6-phosphogluconate, tryptophan, xanthurenic acid, pail⁻nitric acid, ureidosuccinic acid, alpha-d-glucose, glucosamine 6-phosphate, 1-dihydroorotic acid, carnosine, urea, deoxyinosine, imidazole, 3-hydroxybutanoate, glucose 6-phosphate, dGTP, 5-hydroxytryptophan, 2-deoxyglucose-6-phosphate, 6-phosphogluconate, LPA 18:1, LPA 18:2, LPC 20:3, LPC 20:0, LPA 16:1, LPC 22:0, LPC 20:1, PS 18:0/18:1, LPE 22:0, LPC 22:5, PG 18:0/20:4, PS 16:1/18:2:2, PS 16:1/18:1:2, PG 18:0/20:4:2, LPC 17:0, CE 16:0, LPC 24:1, PS 16:0/18:2, LPE 16:1, LPC 24:0, CER 14:0, PG 16:0/18:0:2, PS 18:1/18:2:2, LPE 24:0, PS 16:0/16:1:2, PS 18:0/18:1:2, PG 16:0/18:0, PG 16:0/18:1, PG 16:0/18:1:2, PS 18:0/18:2, LPC 20:4, LPC 16:1, PC 16:0/16:0, PS 18:0/18:0, PS 16:1/18:0:2, LPC 22:6, PG 18:0/18:1, CE 22:4, PG 18:0/18:1, PA 18:0/20:3, PG 18:0/18:2, LPC 15:0, PC 14:0/18:2, CE 18:1, PA 18:1/18:2, PG 16:0/20:3, PG 18:0/18:2, CE 17:0, PG 18:0/18:0, PG 16:0/20:2 , PS 16:0/20:3, FFA 18:2, FFA 18:0, PG 18:1/18:1, CE 18:0, PG 16:1/18:1, PS 16:0/18:0, PA 18:0/18:2, PG 18:1/18:2 , PS 16:0/18:1, LPC 18:1, PG 16:0/18:2, PE P-18:1/20:4, HCER 18:0, Cholesterol, CE 18:3, LPC 14:0, PG 16:0/18:2, PG 16:0/20:2, PG 16:0/20:3, DAG 16:1/18:2, PG 16:0/16:0, PE 18:2/20:2, PG 18:1/18:2, LPE 18:3, LPC 22:1, PE P-18:0/20:4, CE 18:2, PG 18:2/18:2, PS 16:1/18:0, HCER 20:0, FFA 18:1, PG 16:1/18:0:2, LPC 18:2, FFA 16:0, PS 16:0/18:0:2, CE 20:4, PC 16:0/14:0, CE 22:5, LCER 22:0, PC 14:0/16:1, CER 22:0, PG 16:1/18:0, CER 24:1, PC 14:0/18:1, SM d18:1/12:0, PE P-18:1/22:6, FFA 22:2, PE 16:0/16:0, PI 14:0/18:1, LPC 18:3, PE P-18:0/22:6, LPC 18:0, FFA 12:0, PC 12:0/18:2, PG 16:1/18:2:2, FFA 20:4, PG 16:1/18:1:2, CE 22:6, PC 18:0/14:0, FFA 17:0, PC 14:0/18:3, CE 20:3, SM d18:1/14:1, PE P-18:2/20:4, PE P-18:1/22:5, FFA 14:0, PE 18:0/15:0, HCER 20:1, PC 14:0/14:0, TAG 52:2/FA16:0, PE P-16:0/22:6, LPC 20:5, PG 16:0/16:1, FFA 18:4, PE P-18:2/22:6, FFA 20:0, PE P-16:0/16:1, FFA 15:0, PC 18:0/18:0, and HCER 14:0, may be used to determine whether a subject has an increased risk of tumor recurrence after radiation therapy. In certain embodiments, a subject is determined to have an increased risk of tumor recurrence after radiation therapy, or the subject's level of each metabolite and lipid is considered altered as compared to its normal level, when the subject's level of geranyl pyrophosphate, glucose-1-phosphate, 3-hydroxy-3-methylglutaryl-CoA, LPA 18:0, LPA 16:0, and LPC 20:2 is each higher than the respective normal level, and the subject's level of CER 24:0 and LPI 16:1 is each lower than the respective normal level; and one or more of the following is determined: the subject's level of A IC AR is higher than the normal level, the subject's level of carbamoyl phosphate is higher than the normal level, the subject's level of uridine triphosphate is higher than the normal level, the subject's level of fructose is higher than the normal level, the subject's level of 1,6-bisphosphonate is higher than the normal level, the subject's level of 6-phosphogluconate is higher than the normal level, the subject's level of palmitic acid is higher than the normal level, the subject's level of glucosamine 6-phosphate is higher than the normal level, the subject's level of 1-dihydroorotic acid is higher than the normal level, the subject's level of carnosine is higher than the normal level, the subject's level of urea is higher than the normal level, the subject's level of imidazole is higher than the normal level, the subject's level of 3-hydroxybutanoate is higher than the normal level, the subject's level of glucose 6-phosphate is higher than the normal level, the subject's level of 2-deoxyglucose-6-phosphate is higher than the normal level, the subject's level of 6-phosphogluconate is higher than the normal level, the subject's level of taurine is lower than the normal level, the subject's level of n-acetylornithine is lower than the normal level, the subject's level of tryptophan is lower than the normal level, the subject's level of xanthurenic acid is lower than the normal level, the subject's level of ureidosuccinic acid is lower than the normal level, the subject's level of alpha-d-glucose is lower than the normal level, the subject's level of deoxyinosine is lower than the normal level, the subject's level of dGTP is lower than the normal level, the subject's level of 5-hydroxytryptophan is lower than the normal level, the subject's level of LPA 18:1 is higher than the normal level, the subject's level of LPA 18:2 is higher than the normal level, the subject's level of LPC 20:3 is higher than the normal level, the subject's level of LPC 20:0 is higher than the normal level, the subject's level of LPC 22:0 is higher than the normal level, the subject's level of LPC 20:1 is higher than the normal level, the subject's level of LPE 22:0 is higher than the normal level, the subject's level of LPC 22:5 is higher than the normal level, the subject's level of LPC 17:0 is higher than the normal level, the subject's level of LPC 24:1 is higher than the normal level, the subject's level of LPE 16:1 is higher than the normal level, the subject's level of LPC 24:0 is higher than the normal level, the subject's level of LYE 24:0 is higher than the normal level, the subject's level of LPC 20:4 is higher than the normal level, the subject's level of LPC 16:1 is higher than the normal level, the subject's level of PC 16:0/16:0 is higher than the normal level, the subject's level of LPC 22:6 is higher than the normal level, the subject's level of LPC 15:0 is higher than the normal level, the subject's level of PC 14:0/18:2 is higher than the normal level, the subject's level of LPC 18:1 is higher than the normal level, the subject's level of HCER 18:0 is higher than the normal level, the subject's level of LPC 14:0 is higher than the normal level, the subject's level of DAG 16:1/18:2 is higher than the normal level, the subject's level of LPE 18:3 is higher than the normal level the subject's level of LPC 22:1 is higher than the normal level, the subject's level of HCER 20:0 is higher than the normal level, the subject's level of LPC 18:2 is higher than the normal level, the subject's level of PC 16:0/14:0 is higher than the normal level, the subject's level of PC 14:0/16:1 is higher than the normal level, the subject's level of PC 14:0/18:1 is higher than the normal level, the subject's level of SM d18:1/12:0 is higher than the normal level, the subject's level of PE 16:0/16:0 is higher than the normal level, the subject's level of LPC 18:3 is higher than the normal level, the subject's level of LPC 18:0 is higher than the normal level, the subject's level of PC 12:0/18:2 is higher than the normal level, the subject's level of PC 18:0/14:0 is higher than the normal level, the subject's level of PC 14:0118:3 is higher than the normal level, the subject's level of SM d18:1/14:1 is higher than the normal level, the subject's level of PE 18:0/15:0 is higher than the normal level, the subject's level of HCER 20:1 is higher than the normal level, the subject's level of PC 14:0/14:0 is higher than the normal level, the subject's level of LPC 20:5 is higher than the normal level, the subject's level of PE P-16:0/16:1 is higher than the normal level, the subject's level of PC 18:0/18:0 is higher than the normal level, the subject's level of HCER 14:0 is higher than the normal level, the subject's level of LPA 16:1 is lower than the normal level, the subject's level of PS 18:0/181 is lower than the normal level, the subject's level of PG 18:0/20:4 is lower than the normal level, the subject's level of PS 16:1/18:2:2 is lower than the normal level, the subject's level of PS 16:1/18:1:2 is lower than the normal level, the subject's level of PG 18:0/20:4:2 is lower than the normal level, the subject's level of CE 16:0 is lower than the normal level, PS 16:0/18:2 is lower than the normal level, the subject's level of CER 14:0 is lower than the normal level, the subject's level of PG 16:0/18:0:2 is lower than the normal level, the subject's level of PS 18:1/18:2:2 is lower than the normal level, the subject's level of PS 16:0/16:1:2 is lower than the normal level, the subject's level of PS 18:0/18:1:2 is lower than the normal level, the subject's level of PG 16:0/18:0 is lower than the normal level, the subject's level of PG 16:0/18:1 is lower than the normal level, the subject's level of PG 16:0/18:1:2 is lower than the normal level, the subject's level of PS 18:0/18:2 is lower than the normal level, the subject's level of PS 18:0/18:0 is lower than the normal level, the subject's level of PS 16:1/18:0:2 is lower than the normal level, the subject's level of PG 18:0/18:1 is lower than the normal level, the subject's level of CE 22:4 is lower than the normal level, the subject's level of PG 18:0/18:1 is lower than the normal level, the subject's level of PA 18:0/20:3 is lower than the normal level, the subject's level of PG 18:0/18:2 is lower than the normal level, the subject's level of CE 18:1 is lower than the normal level, the subject's level of PA 18:1/18:2 is lower than the normal level, the subject's level of PG 16:0/20:3 is lower than the normal level, the subject's level of PG 18:0/18:2 is lower than the normal level, the subject's level of CE 17:0 is lower than the normal level, the subject's level of PG 18:0/18:0 is lower than the normal level, the subject's level of PG 16:0/20:2 is lower than the normal level, the subject's level of PS 16:0/20:3 is lower than the normal level, the subject's level of FFA 18:2 is lower than the normal level, the subject's level of FFA 18:0 is lower than the normal level, the subject's level of PG 18:1/18:1 is lower than the normal level, the subject's level of CE 18:0 is lower than the normal level, the subject's level of PG 16:1/18:1 is lower than the normal level, the subject's level of PS 16:0/18:0 is lower than the normal level, the subject's level of PA 18:0/18:2 is lower than the normal level, the subject's level of PG 18:1/18:2 is lower than the normal level, the subject's level of PS 16:0/18:1 is lower than the normal level, the subject's level of PG 16:0/18:2 is lower than the normal level, the subject's level of PE P-18:1/20:4 is lower than the normal level, the subject's level of cholesterol is lower than the normal level, the subject's level of CE 18:3 is lower than the normal level, the subject's level of PG 16:0/18:2 is lower than the normal level, the subject's level of PG 16:0/20:2 is lower than the normal level, the subject's level of PG 16:0/20:3 is lower than the normal level, the subject's level of PG 16:0/16:0 is lower than the normal level, the subject's level of PE 18:2/20:2 is lower than the normal level, the subject's level of PG 18:1/18:2 is lower than the normal level, the subject's level of PE P18:0/20:4 is lower than the normal level, the subject's level of CE 18:2 is lower than the normal level, the subject's level of PG 18:2/18:2 is lower than the normal level, the subject's level of PS 16:1/18:0 is lower than the normal level, the subject's level of FFA 18:1 is lower than the normal level, the subject's level of PG 16:1/18:0:2 is lower than the normal level, the subject's level of FFA 16:0 is lower than the normal level, the subject's level of PS 16:0/18:0:2 is lower than the normal level, the subject's level of CE 20:4 is lower than the normal level, the subject's level of CE 22:5 is lower than the normal level, the subject's level of LCER 22:0 is lower than the normal level, the subject's level of CER 22:0 is lower than the normal level, the subject's level of PG 16:1/18:0 is lower than the normal level, the subject's level of CER 24:1 is lower than the normal level, the subject's level of PE P-18:1/22:6 is lower than the normal level, the subject's level of HA 22:2 is lower than the normal level, the subject's level of PI 14:0/18:1 is lower than the normal level, the subject's level of PE P-18:0/22:6 is lower than the normal level, the subject's level of FIFA 12:0 is lower than the normal level, the subject's level of PG 16:1/18:2:2 is lower than the normal level, the subject's level of FFA 20:4 is lower than the normal level, the subject's level of PG 16:1/18:1:2 is lower than the normal level, the subject's level of CE 22:6 is lower than the normal level, the subject's level of FFA 17:0 is lower than the normal level, the subject's level of CE 20:3 is lower than the normal level, the subject's level of PE P-18:2/20:4 is lower than the normal level, the subject's level of PE P-18:1/22:5 is lower than the normal level, the subject's level of FFA 14:0 is lower than the normal level, the subject's level of TAG 52:2/FA16:0 is lower than the normal level, the subject's level of PE P-16:0/22:6 is lower than the normal level, the subject's level of PG 16:0/16:1 is lower than the normal level, the subject's level of FFA 18:4 is lower than the normal level, the subject's level of PE P-18:2/22:6 is lower than the normal level, the subject's level of FFA 20:0 is lower than the normal level, and the subject's level of FFA 15:0 is lower than the normal level.

In embodiments of the invention, the component profile comprises metanephrine, tryptophan, xanthurenic acid, pantothenate, LPA 18:0, DAG 16:0/18:0, LPA 16:0, and DAG 18:1/18:1. In some embodiments, the component profile comprising metanephrine, tryptophan, xanthurenic acid, pantothenate, LPA 18:0, DAG 16:0/18:0, LPA 16:0, and DAG 18:1/18:1, may be used to determine whether a subject has an increased risk of one or more late effects, such as rectal toxicity or urinary toxicity, after radiation therapy. In certain embodiments, a subject is determined to have an increased risk of late effects after radiation therapy, or the subject's level of each metabolite and lipid is considered altered as compared to its normal level, when the subject's level of LPA18:0 and LPA 16:0 is each higher than the respective normal level, and the subject's level of metanephrine, tryptophan, xanthurenic acid, pantothenate, DAG 16:0/18:1, and DAG 18:1/18:1 is each lower than the respective normal level.

In embodiments of the invention, the component profile comprises metanephrine, tryptophan, xanthurenic acid, pantothenate, LPA 18:0, DAG 16:0/18:0, LPA 16:0, and DAG 18:1/18:1, and one or more components selected from the following: 4-pyridoxate, methylthioadenosine, tryptophan, phenylacetyl-1-glutamine, ADP, n-acetyl ornithine, hydroxyisocaproic acid, LPI 16:0, LPA 18:2, LPA 18:1, PI 16:1-18:1:2, CE 201, LPA 16:1, FFA 202, PC 180/225, and PE 180:225. In some embodiments, the component profile comprising metanephrine, tryptophan, xanthurenic acid, pantothenate, LPA 18:0, DAG 16:0/18:0, LPA 16:0, and DAG 18:1/18:1, and one or more components selected from 4-pyridoxate, methylthioadenosine, tryptophan, phenylacetyl-1-glutamine, ADP, n-acetyl ornithine, hydroxyisocaproic acid, LPI 16:0, LPA 18:2, LPA 18:1, PI 16:1-18:1:2, CE 201, LPA 16:1, FFA 202, PC 180/225, and PE 180:225, may be used to determine whether a subject has an increased risk of one or more late effects, such as rectal toxicity or urinary toxicity, after radiation therapy. In certain embodiments, a subject is determined to have an increased risk of late effects after radiation therapy, or the subject's level of each metabolite and lipid is considered altered as compared to its normal level, when the subject's level of LPA18:0 and LPA 16:0 is each higher than the respective normal level, and the subject's level of metanephrine, tryptophan, xanthurenic acid, pantothenate, DAG 16:0/18:1, and DAG 18:1/18:1 is each lower than the respective normal level; and one or more of the following is determined: the subject's level of 4-pyridoxate is lower than the normal level, the subject's level of methylthioadenosine is lower than the normal level, the subject's level of tryptophan is lower than the normal level, the subject's level of phenylacetyl-l-glutamine is lower than the normal level, the subject's level of ADP is lower than the normal level, the subject's level of n-acetylornithine is lower than the normal level, the subject's level of hydroxyisocaproic acid is lower than the normal level, the subject's level of LPI 16:0 is lower than the normal level, the subject's level of LPA 18:2 is higher than the normal level, the subject's level of LPA 18:1 is higher than the normal level, the subject's level of PI 16:1-18:1:2 is lower than the normal level, the subject's level of CE 201 is higher than the normal level, the subject's level of LPA 16:1 is higher than the normal level, the subject's level of FFA 202 is higher than the normal level, the subject's level of PC 180/225 is lower than the normal level, and the subject's level of PE 180:225 is lower than the normal level.

### EXAMPLES

### Example 1

Patients were enrolled at MedStar-Georgetown University Hospital into IRB protocol 2012-1175; an approved quality of life clinical trial. The protocol permits longitudinal collection of clinical samples, symptom monitoring and quality of life data which have contributed to interim published reports of clinical outcomes including GU and GI acute and late effects. This study population was a part of ongoing recruitment of PC patients coming in through the referral network to MedStar-Georgetown University Hospital (MGUH). The study participants included men of varying races aged 35-70 years, residing in Washington DC and surrounding areas, who were diagnosed with localized prostate cancer by biopsy. Patients were recruited from the Departments of Radiation Medicine and Urology at the MGUH.

Regarding technical aspects of stereotactic body radiation therapy (SBRT) treatment planning and radiation delivery, briefly, ultrasound guided placement of gold fiducial markers is performed 2 or more weeks prior to thin cut CT and high resolution MRI imaging. The clinical target volume (CTV) includes the prostate and proximal seminal vesicles, to the bifurcation. The prescribed doses of 35-36.25 Gy are delivered in five fractions of 7-7.25 Gy over 2 weeks. Symptom management medications were prescribed based on the treating physician's clinical judgment and urinary symptoms were managed with alpha-adrenergic antagonists and bothersome bowel symptoms were managed with anti-diarrheal medication (loperamide).

### Example 2

The objective of this study was to employ a high through put metabolomics approach for delineating a biomarker panel predictive of radiation induced adverse effects in patients treated for prostate cancer. Such biomarkers aid in early detection of tissue toxicity in cancer patients, so that intervention can be initiated early in patients at risk. Metabolite signatures were developed for prediction of adverse responses to radiation therapy in a cohort of patients undergoing stereotactic body radiation therapy (SBRT) for prostate cancer. Subsets of these patients developed urinary toxicity (UT) (N=8) and rectal toxicity (RT) (N=6).

Individuals sensitive to radiation toxicities carry a biochemical fingerprint that can be identified as a distinct plasma profile. Analysis of banked plasma samples was correlated with clinical outcomes and symptom assessment to identify markers of genitourinary and gastrointestinal late effects for future validation in a larger clinical population. Using stable isotope labeling multiple reaction monitoring based molecular phenotyping approach, high accuracy predictive algorithms were developed for recurrence, urinary toxicity, and rectal toxicity episodes in this cohort. This analysis was performed using the pre-radiation samples from this set of patients. The panels can be useful to predict late effects of radiation therapy and lay the foundation for the development of strategies by which toxicity may be detected at an early stage and mitigated with intervention therapies.

### Example 2A

Stable isotope labeled multiple reaction monitoring mass spectrometry (SID-MRM-MS) was used for quantitation of 350 metabolites. Metabolite extraction was performed using 25 μL of plasma sample taken from the patients described in Example 1. The plasma sample was mixed with 175 μL of 40% acetonitrile in 25% methanol and 35% water containing internal standards [stable isotope labeled). The samples were incubated on ice for 10 minutes and centrifuged at 14,000 rpm at 4° C. for 20 minutes. The supernatant was transferred to a fresh tube and used for UPLC-QQQ -MS analysis. Each plasma sample (2 μL) was injected onto a reverse-phase CSH C18 1.7 μM 2.1×100 mm column using an Acquity UPLC online with a triple quadrupole MS (Xevo TQ-S, Waters Corporation, USA) G2-QTOF system operating in the MRM mode.

Following data pre-processing and ion annotation, the m/z values of the measured metabolites were normalized with log transformation that stabilizes variance, followed by quantile normalization to achieve uniform empirical distribution of intensities (measure of metabolite abundance) across samples.

After the data pre-processing and ion annotation, the m/z values of the measured metabolites from tissue samples were normalized with log transformation that stabilized the variance, followed by quantile normalization to make the empirical distribution of intensities the same across samples. Differential expression between various patient groups was assessed using analysis of variance (ANOVA). Multiple comparisons were adjusted using the Bonferroni correction. The heat maps were generated for the significant metabolites using the log 2 transformed values of fold changes and hierarchically clustered by Pearson correlation.

Among these differentially expressed metabolites identified, feature selection was performed using a regularized learning technique, which uses the least absolute shrinkage and selection operator (LASSO) penalty. Differential expression between various patient groups was assessed using t-test constrained by p-value <0.05. Among these differentially expressed metabolites, each m/z value was scored for annotation against the HMDB, Metlin, MMCD and Lipid Maps databases within a 5 ppm mass tolerance.

After the data pre-processing and ion annotation, the m/z values of the measured metabolites from plasma samples were normalized with log transformation that stabilized the variance, followed by quantile normalization to make the empirical distribution of intensities the same across samples. Differential expression between various patient groups was assessed using analysis of variance (ANOVA). Multiple comparisons were adjusted using the Bonferroni correction.

Among these differentially expressed metabolites identified, feature selection was performed using a receiver operating characteristic (ROC)-regularized learning technique, which uses the least absolute shrinkage and selection operator (LASSO) penalty. The regularization path was obtained over a grid of values for the tuning parameter lambda through 10-fold cross-validation. Then the optimal value of the tuning parameter lambda, obtained by the cross-validation procedure was used to fit the model. Finally, all the features with non-zero coefficients were retained as the candidate biomarker panel. This technique is known to reduce overfitting and variance in classification.

The classification performance of the biomarker panel was assessed using the area under the ROC (receiver operating characteristic) curve (AUC). The ROC curve can be understood as a plot of the probability of classifying correctly positive samples against the rate of incorrectly classifying true negative samples. Therefore, the AUC measure of an ROC plot is a measure of predictive accuracy. Due to the perfect separation for the classification, the panel was also evaluated using the hidden logistic regression model with the maximum estimated likelihood (MEL) estimator, and the AUC scores were similar. The individual markers were also analyzed, and the AUC score was estimated for the regression with each marker, all of them showing high discriminative value for distinguishing “high” vs “low” and “recur” vs “low” patient group, to rule out correlation with the patients' hormone therapy status.

Leveraging the longitudinally collected clinical outcome data, a retrospective outcome analysis was performed on this cohort to determine if there was a differentiation between the low risk, high risk and recurrence categories of patients by comparing their metabolic profiles at baseline. The abundance measurements for metabolites were expressed as intensity units that were initially normalized using log transformation and quantile normalization.

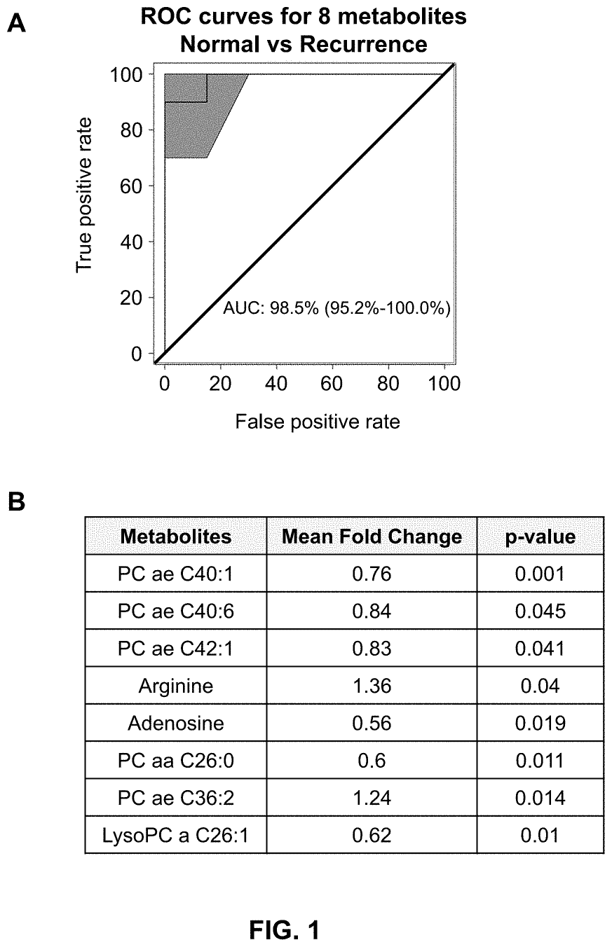

The cohort of SBRT treated patients is shown in FIG. 1, Panels A and B. Of 105 patients, 10 developed biochemical recurrences with an average time of 18 months. To develop a predictive panel of recurrence, the pre-radiation plasma metabolite profiles were compared to a sub-set of patients who remained cancer free during this time. The panel was adjusted for age, PSA levels and Gleason's grade. A sub-set analyses was performed in patients not receiving hormone therapy to rule out the influence of hormone therapy on the marker panel, in high risk and recurrence patient categories.

### Example 2B

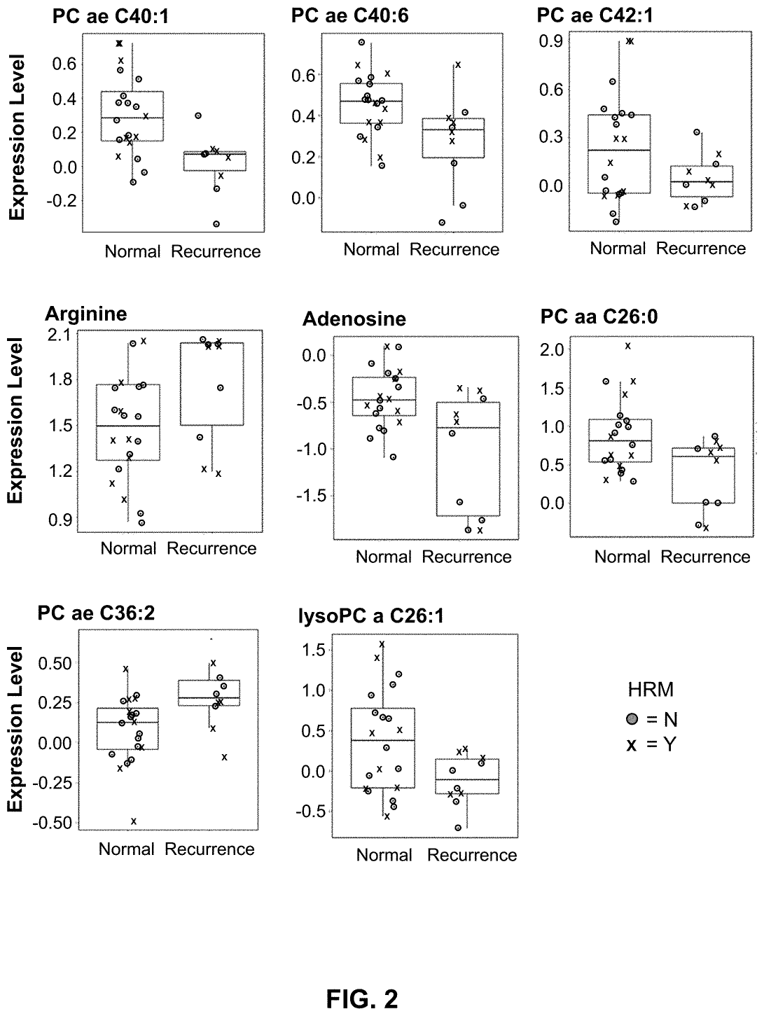

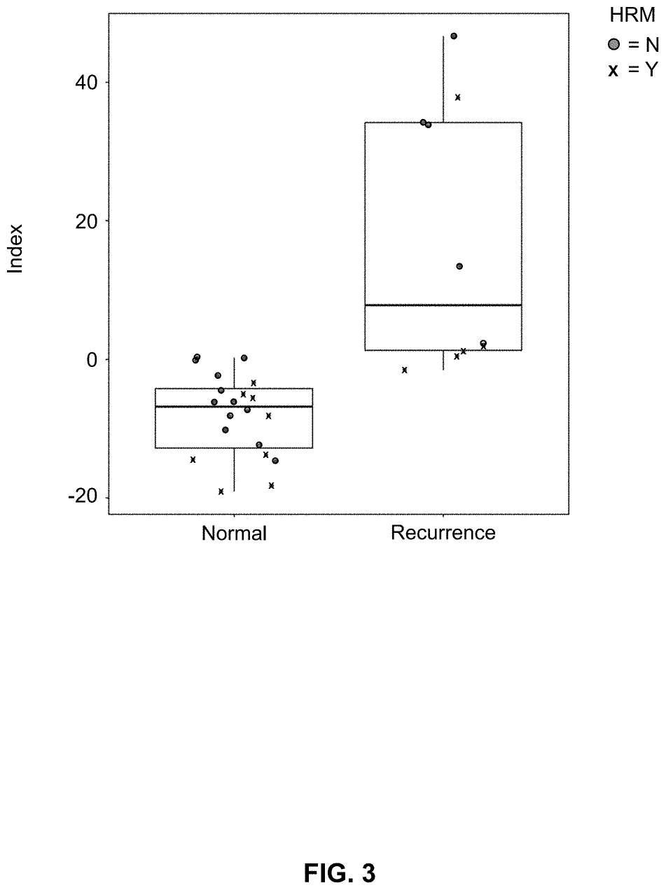

Applying the sampling and analytical procedure discussed in Example 2, an eight metabolite panel with an AUC >98% (FIG. 1, Panels A and B; Table 1) was able to accurately discriminate between those who reported recurrence episodes (N=10) as compared to those who remained cancer free (N=20) during the follow up period. Differential expression of these biomarkers in the two comparative groups was visualized as box plots (FIG. 2). Finally, the resulting combined classifier allowed the development of a plasma metabolite index (PMI), which was obtained by mapping the 8 metabolites to the hyperplane that maximizes the margin between different groups (FIG. 3). Based on the model with the biomarker panel, the eight-member PMI (8PMI) helps identify the degree to which individuals are ‘at-risk’ of an outcome (recurrence, rectal or bladder late effects). The natural log of odds in the model was transformed to 0-100 index value using a linear mapping.

TABLE 1
Biomarker panel predictive of tumor recurrence in PC patients.

| Metabolites | Mean Fold Change | p-value |
|---|---|---|
| PC ae C40:1 | 0.76 | 0.001 |
| PC ae C40:6 | 0.84 | 0.045 |
| PC ae C42:1 | 0.83 | 0.041 |
| Arginine | 1.36 | 0.04 |
| Adenosine | 0.56 | 0.019 |
| PC aa C26:0 | 0.6 | 0.011 |
| PC ae C36:2 | 1.24 | 0.014 |
| LysoPC a C26:1 | 0.62 | 0.01 |

### Example 2C

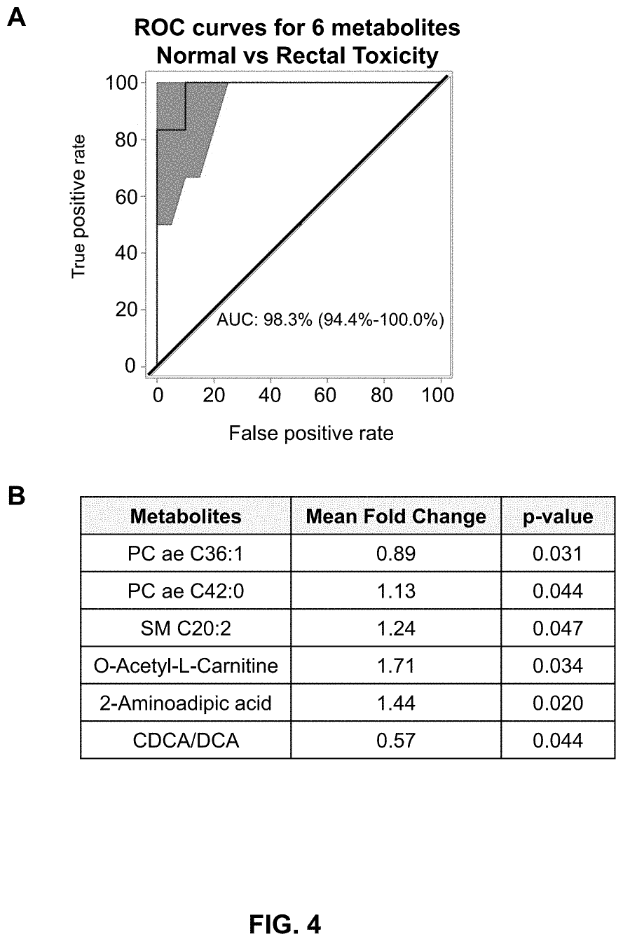

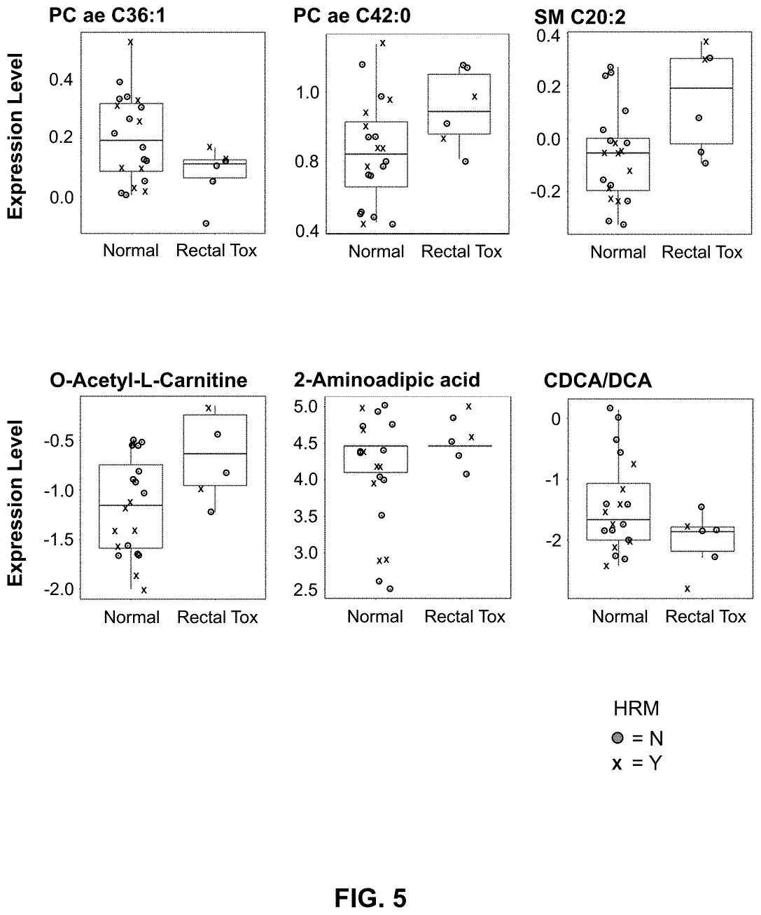

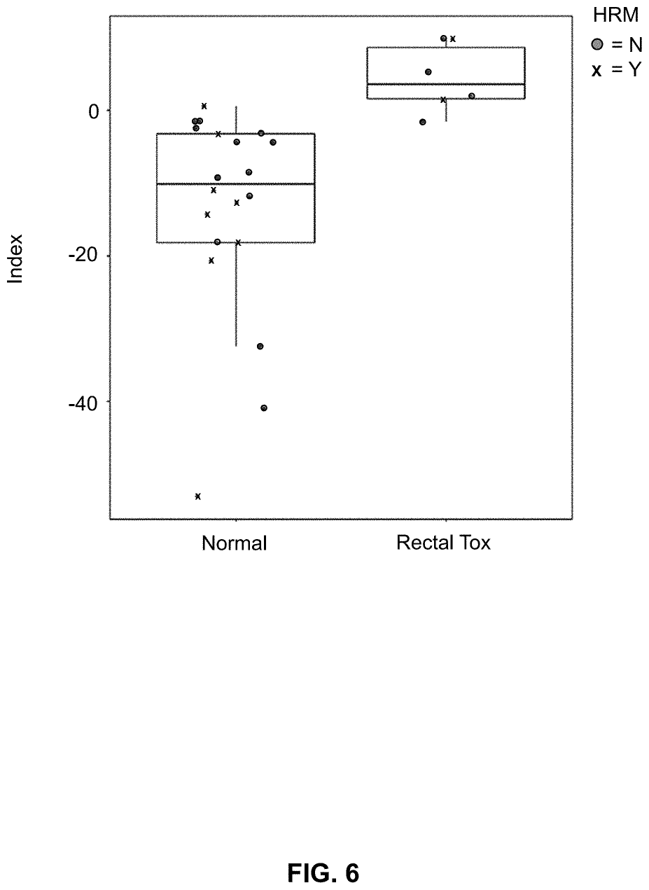

Applying the sampling and analytical procedure discussed in Example 2, a predictive biomarker panels of adverse outcomes of radiation therapy was also developed. A retrospective outcome analysis was performed on a sub-set of patients who reported rectal toxicity (N=6) by comparing their pre-radiation plasma metabolite profiles with a randomly selected sub-set of prostate cancer patients who remained normal during the post-radiation monitoring period. A six metabolite biomarker panel yielded an AUC of 98.3% (FIG. 4, Panels A and B; Table 2). The relative abundance of biomarkers in the two comparative groups is illustrated in FIG. 5 while the six-member PMI (6PMI) (FIG. 6), helped stratify patients who later developed rectal toxicity from those who did not develop radiation induced adverse symptoms, underscoring the power of this technology.

TABLE 2
Six metabolite panel predictive of rectal toxicity.

| Metabolites | Mean Fold Change | p-value |
|---|---|---|
| PC ae C36:1 | 0.89 | 0.031 |
| PC ae C42:0 | 1.13 | 0.044 |
| SM C20:2 | 1.24 | 0.047 |
| O-Acetyl-L-Carnitine | 1.71 | 0.034 |
| 2-Aminoadipic Acid | 1.44 | 0.020 |
| CDCA/DCA | 0.57 | 0.044 |

### Example 2D

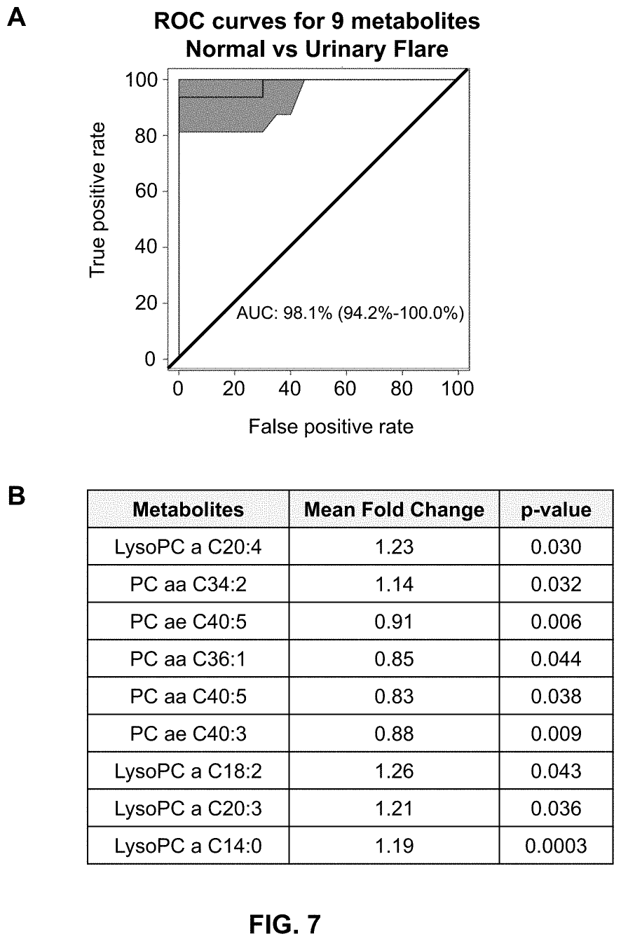

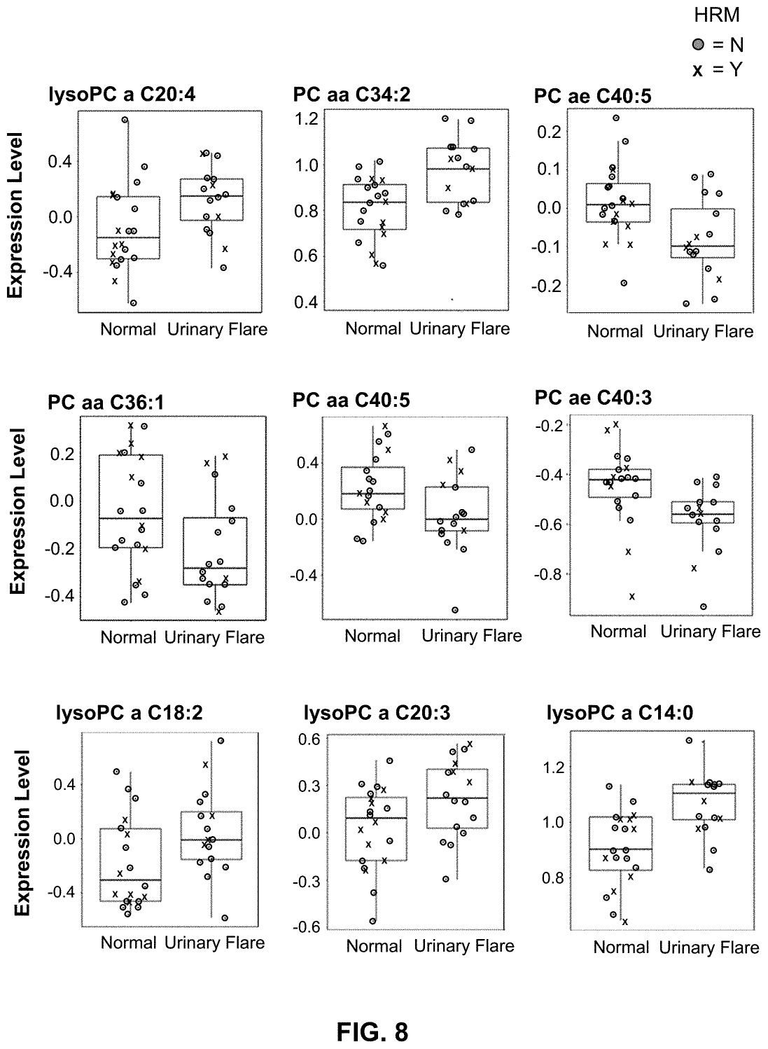

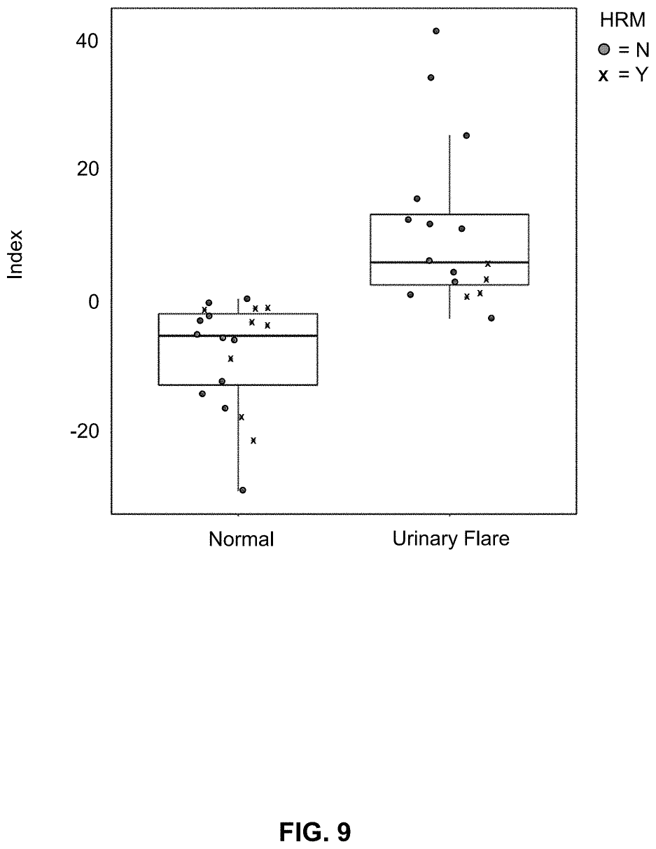

Applying the sampling and analytical procedure discussed in Example 2, using LASSO, a nine metabolite panel with AUC >98% (FIG. 7, Panels A and B; Table 3) was established for patients' pre-radiation profiles and those patients who reported urinary toxicity. The pattern of expression of individual markers was visualized as a box plot (FIG. 8), while a nine-member PMI (9PMI) that was developed using logistic regression helped stratify the two comparative groups unambiguously (FIG. 9).

The classification algorithm was developed that would be predictive of urinary flare (N=8). It is noteworthy that some of the patients who developed urinary flare also developed tumor recurrence.

TABLE 3
Biomarker panel predictive of urinary flare

| Metabolites | Mean Fold Change | p-value |
|---|---|---|
| LysoPC a C20:4 | 1.23 | 0.030 |
| PC aa C34:2 | 1.14 | 0.032 |
| PC ae C40:5 | 0.91 | 0.006 |
| PC aa C36:1 | 0.85 | 0.044 |
| PC aa C40:5 | 0.83 | 0.038 |
| PC ae C40:3 | 0.88 | 0.009 |
| LysoPC a C18:2 | 1.26 | 0.043 |
| LysoPC a C20:3 | 1.21 | 0.036 |
| LysoPC a C14:0 | 1.19 | 0.0003 |

### Example 3

Metabolomics and lipidomics analyses were performed using a QTRAP® 5500 LC-MS/MS system on plasma samples from the patients discussed in Example 1.

### Methods

For the metabolomics analysis, 50 μL of plasma sample was transferred to a glass tube for extraction, and 0.9 mL of H₂O was added. Then, 2 mL of methanol and 0.9 mL of dichloromethane (DCM) were added, and the sample was mixed gently but thoroughly for 5 seconds. The samples were incubated at room temperature for 30 minutes without disturbance. Then 1 mL of water and 2 mL of chloroform were added to the sample and the sample was centrifuged at 2000 X for 20 minutes. The upper aqueous layer was collected and refrigerated in −80° C. for 5 hours and then lyophilized. The lyophilized samples were reconstituted in 0.2 mL of 1:1 acetonitrile/water containing 200 ng/mL of debrisoquine (DBQ) as internal standard for positive mode and 200 ng/mL of taurine-d4 as internal standard for negative mode. The samples were centrifuged at 13,000 rpm for 20 min at 4° C. just prior to analysis and the supernatant was transferred to MS vials for LC-MS analysis. 5 μL of the prepared sample was injected onto a Kinetex 2.6 μm 100 Å 100×2.1 mm (Phenomenex) using SIL-30 AC auto sampler (Shimazdu) connected with a high flow LC-30AD solvent delivery unit (Shimazdu) and CBM-20A communication bus module (Shimazdu) online with QTRAP 5500 LC-MS/MS system (Sciex, MA, USA) operating in positive and negative ion mode. A binary solvent comprising of water with 0.1% formic acid (Solvent A) and acetonitrile with 0.1% formic acid (Solvent B) was used. The extracted metabolites were resolved at 0.2 mL/min flow rate starting with 100% of Solvent A and holding for 2.1 minutes; moving to 5% of Solvent A over a time period of 12 minutes and holding for 1 minute; and equilibrating to initial conditions over a time period of 7 minutes using auto sampler temperature 15° C. and oven temperature 30° C. Source and gas setting for the method were as shown in Table 4.

TABLE 4
Source and gas settings used in processing
samples for metabolomics analysis

| Parameter | Setting |
|---|---|
| Curtain gas | 35 |
| CAD gas | Medium |
| Ion spray voltage, positive mode | 2.5 kV |
| Ion spray voltage, negative mode | −4.5 kV |
| Temperature | 400° C. |
| Nebulizing gas | 60 |
| Heater gas | 70 |

The data were normalized to internal standard area and processed using MultiQuant 3.0.3 (Sciex). The column was conditioned using the pooled QC samples initially and were also injected periodically (after every 10 sample injections) to monitor shifts in signal intensities and retention time as measures of reproducibility and data quality of the LC-MS data. NIST plasma sample (after every 20 samples) prepared in the same manner were run to check the instrumental variance. In addition, blank solvent was run between set of samples (after every 10 samples before and after pooled QC samples) to minimize carry-over effects.

For the lipidomics analysis, 20 μL of each plasma sample was dissolved in 100 μL of chilled isopropanol containing internal standards, and then vortexed. The samples were kept on ice for 30 minutes and then incubated at −20° C. for 2 hours for complete protein precipitation. The samples were then centrifuged at 13,000 rpm for 20 minutes at 4° C. The supernatant was transferred to MS vial for LC-MS analysis. Five μL of each sample was injected onto a Xbridge amide 3.5 μm, 4.6×100 mm (waters) using SIL-30 AC auto sampler (Shimazdu) connected with a high flow LC-30AD solvent delivery unit (Shimazdu) and CBM-20A communication bus module (Shimazdu) online with QTRAP 5500 (Sciex, MA, USA) operating in positive and negative ion mode. For the resolution, a binary solvent was used, comprising of acetonitrile/water 95/5 with 10 mM ammonium acetate as Solvent A and acetonitrile/water 50/50 with 10 mM ammonium acetate as Solvent B. Lipids were resolved at 0.7 mL/min flow rate, initial gradient conditions started with 100% of Solvent A, shifting towards 99.9% of Solvent A over a time period of 3 minutes, 94% of Solvent A over a time period of 3 minutes, and 25% of solvent A over a period of 4 minutes. Finally, lipids were washed with 100% of Solvent B for 6 minutes and equilibrating to initial conditions (100% of solvent A) over a time period of 6 minutes using auto sampler temperature 15° C. and oven temperature 35° C. Source and gas setting were as shown in Table 5.

TABLE 5
Source and gas settings used in processing
samples for lipidomics analysis

| Parameter | Setting |
|---|---|
| Curtain gas | 30 |
| CAD gas | Medium |
| Ion spray voltage, positive mode | 5.5 kV |
| Ion spray voltage, negative mode | −4.5 kV |
| Temperature | 550° C. |
| Nebulizing gas | 50 |
| Heater gas | 60 |

The data were normalized to internal standard area for each class of lipid and processed using MultiQuant 3.0.3 (Sciex). The quality and reproducibility of LC-MS data was ensured using a number of measures. The column was conditioned using the pooled QC samples initially and were also injected periodically (after every 10 sample injections) to monitor shifts in signal intensities and retention time as measures of reproducibility and data quality of the LC-MS data.

The raw LC-MS data were initially normalized by internal standards. The pre-processing was followed up by quality control procedure which involves the calculation of the normalized intensities based relative standard deviation (RSD) for each feature. The features with more than 15% of coefficient of variation (CV) were filtered out. The features with missing value more than 20% were also filtered out. For less than 20% of missing value, those features were imputed by half of the minimum positive value in the original data. For the lipidomics panel, 9 features were filtered out based on missing value threshold and 49 filtered out for QC-RSD. In the metabolomics panel, 188 features were removed based on missing value criteria and 18 features filtered out for QC-RSD >15%. This resulted in 144 reliable metabolites and 751 lipids.

The remaining high quality peak intensities were then QC-RLSC normalized and summarized for quantification. The p-values for binary comparisons were calculated by two-tailed unpaired Student t-test, whereas p values of less than 0.05 exclusively were considered significant. Furthermore, p-values were corrected for multiple testing using Benjamin-Hochberg procedure which limits the false discovery rate (FDR).

All the FDR adjusted p-value significant features were considered as candidate biomarkers for the feature selection. Feature selection was then performed using a ROC regularized learning technique, which uses the LASSO penalty. The regularization path was obtained over a grid of values for the tuning parameter lambda through tenfold cross validation. Then, the optimal value of lambda, obtained by the cross-validation procedure, was used to fit the model. All the features with non-zero coefficients were retained as the candidate biomarker panel. This technique was known to reduce over-fitting and variance in classification. The classification performance of the biomarker panel was assessed using the area under the ROC curve.

### Results

The subjects who were evaluated in this study are summarized in Table 6.

TABLE 6
Characteristics of subjects who were evaluated in the study.

| | | | PSA (SD) | | Gleason |
|---|---|---|---|---|---|
| Symptoms | N | Age (SD) | [ng/mL] | | Score (SD) |
| Normal | 69 | 68.07 (7.67) | 11.49 | (15.47) | 6.94 (0.94) |
| Tumor recurrence | 10 | 73.10 (9.55) | 14.27 | (8.99) | 7.60 (1.17) |
| Late effects | 15 | 73.66 (7.41) | 5.74 | (3.32) | 7.00 (1.06) |

SD = standard deviation
PSA = Prostate Specific Antigen

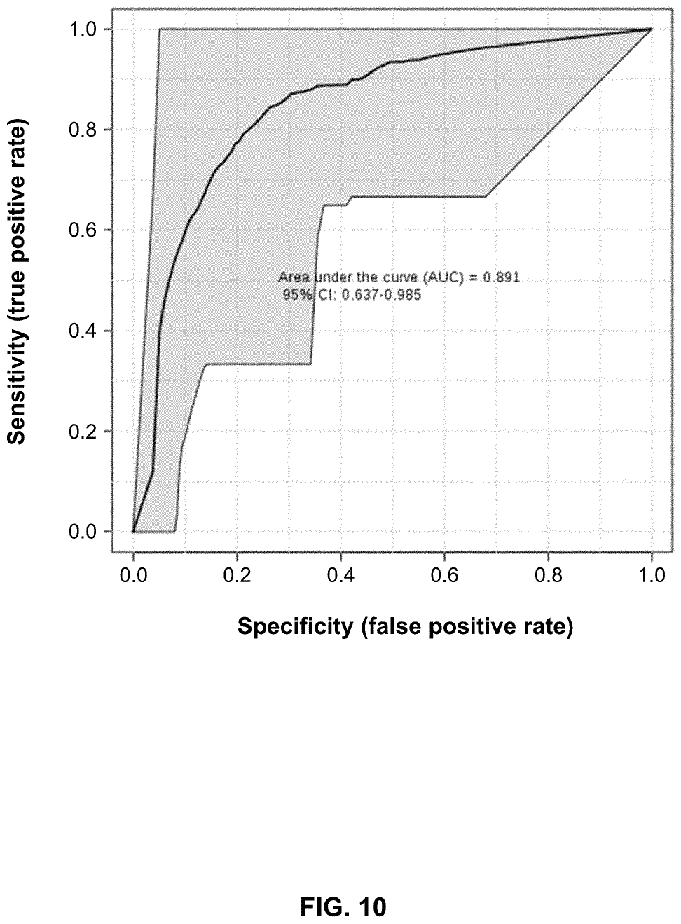

The results showed that a metabolite panel comprising at least geranyl pyrophosphate, glucose-1-phosphate, and 3-hydroxy-3-methylglutaryl-CoA demonstrated predictability of tumor recurrence in subjects (see FIG. 10; Table 7).

TABLE 7
Predictive accuracy of three-metabolite
panel regarding tumor recurrence.

| | | | Specificity^{a} | Sensitivity^{b} |
|---|---|---|---|---|
| Metabolites | AUC | 95% CI | (%) | (%) |
| Geranyl pyrophosphate | 0.891 | 0.637-0.985 | 17.6 | 37.1 |
| Glucose-1-phosphate | | | | |
| 3-hydroxy-3- | | | | |
| methylglutaryl-CoA | | | | |

^{a}Specificity at 95% sensitivity
^{b}Sensitivity at 95% specificity

In addition, other metabolites were identified as exhibiting a fold change between the two comparative groups and could be included in a panel to predict those who are at risk of recurrence (see Table 8).

TABLE 8
Metabolites exhibiting a fold change between
subjects who reported recurrence episodes as
compared to those who remained cancer-free.

| Metabolites | p-value | FDR | Fold Change |
|---|---|---|---|
| Geranyl pyrophosphate | 2.85E−06 | 2.05E−04 | 1.80 ↑ |
| Glucose 1-Phosphate | 1.64E−05 | 7.75E−04 | 2.39 ↑ |
| Taurine | 2.94E−05 | 8.41E−04 | 0.22 ↓ |
| Geranyl-PP0 | 2.89E−06 | 2.05E−04 | 1.80 ↑ |
| AICAR | 3.15E−05 | 8.41E−04 | 2.24 ↑ |
| 3-hydroxy-3-methylglutaryl-CoA | 3.55E−05 | 8.41E−04 | 1.86 ↑ |
| Carbamoyl phosphate | 4.66E−05 | 9.45E−04 | 1.58 ↑ |
| Uridine triphoshate | 1.04E−04 | 1.85E−03 | 2.13 ↑ |
| Fructose 1,6-bisphosphonate | 1.92E−03 | 3.02E−02 | 1.72 ↑ |
| N-acetylornithine | 2.62E−03 | 3.73E−02 | 0.55 ↓ |
| 6-phosphogluconate | 3.11E−03 | 4.01E−02 | 1.55 ↑ |
| Tryptophan | 7.19E−03 | 7.76E−02 | 0.42 ↓ |
| Xanthurenic acid | 7.63E−03 | 7.76E−02 | 0.48 ↓ |
| Palmitic acid | 7.65E−03 | 7.76E−02 | 2.56 ↑ |
| Ureidosuccinic acid | 9.71E−03 | 9.19E−02 | 0.44 ↓ |
| Alpha-d-glucose | 1.06E−02 | 9.42E−02 | 0.66 ↓ |
| Glucosamine 6-phosphate | 1.52E−02 | 1.27E−01 | 1.53 ↑ |
| L-dihydroorotic acid | 2.30E−02 | 1.81E−01 | 1.56 ↑ |
| Carnosine | 3.08E−02 | 2.10E−01 | 2.38 ↑ |
| Urea | 3.35E−02 | 2.10E−01 | 1.51 ↑ |
| Deoxyinosine | 3.38E−02 | 2.10E−01 | 0.72 ↓ |
| Imidazole | 3.39E−02 | 2.10E−01 | 2.56 ↑ |
| 3-hydroxybutanoate | 3.52E−02 | 2.10E−01 | 2.05 ↑ |
| Glucose 6-phosphate | 3.58E−02 | 2.10E−01 | 1.38 ↑ |
| dGTP | 3.69E−02 | 2.10E−01 | 0.81 ↓ |
| 5-hydroxytryptophan | 3.92E−02 | 2.14E−01 | 0.69 ↓ |
| 2-deoxyglucose-6-phosphate | 4.96E−02 | 2.46E−01 | 1.33 ↑ |
| 6-phosphogluconate | 4.98E−02 | 2.46E−01 | 2.03 ↑ |

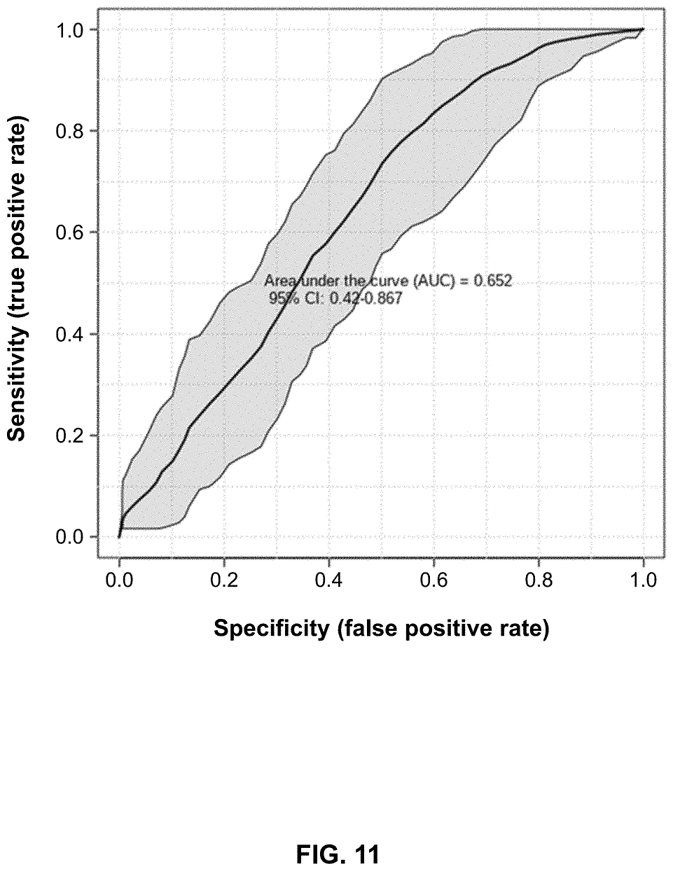

A metabolite panel comprising at least metanephrine, tryptophan, xanthurenic acid, and pantothenate demonstrated yielded an AUC>0.65 (see FIG. 11; Table 9).

TABLE 9
Predictive accuracy of three-metabolite panel regarding late effects.

| | | | Specificity^{a} | Sensitivity^{b} |
|---|---|---|---|---|
| Metabolites | AUC | 95% CI | (%) | (%) |
| Metanephrine | 0.652 | 0.42-0.867 | 23.7 | 9.2 |
| Tryptophan | | | | |
| Xanthurenic acid | | | | |
| Pantothenate | | | | |

^{a}Specificity at 95% sensitivity
^{b}Sensitivity at 95% specificity

Other metabolites were identified as exhibiting a fold change between the two comparative groups and could be included in a panel to predict those who are at risk of late effects (see Table 10).

TABLE 10
Metabolites exhibiting a fold change between subjects who reported
late effects compared to those who did not report late effects.

| | | | Fold | Log2 |
|---|---|---|---|---|
| Metabolites | p-value | FDR | Change | (FC) |
| Metanephrine | 0.002627 | 0.33057 | 0.36389 ↓ | −1.4584 |
| Tryptophan | 0.004656 | 0.33057 | 0.64815 ↓ | −0.6256 |
| Xanthurenic acid | 0.007601 | 0.33885 | 0.6962 ↓ | −0.52242 |
| Pantothenate | 0.010219 | 0.33885 | 0.64877 ↓ | −0.62421 |
| 4-Pyridoxate | 0.01297 | 0.33885 | 0.63927 ↓ | −0.6455 |
| Methylthioadenosine | 0.014676 | 0.33885 | 0.68644 ↓ | −0.5428 |
| Tryptophan | 0.016704 | 0.33885 | 0.69915 ↓ | −0.51633 |
| Phenylacetyl-1-glutamine | 0.030073 | 0.51732 | 0.7144 ↓ | −0.48519 |
| ADP | 0.032788 | 0.51732 | 0.4182 ↓ | −1.2577 |
| N-acetylornithine | 0.038082 | 0.54077 | 0.7258 ↓ | −0.46235 |
| Hydroxyisocaproic acid | 0.044743 | 0.57759 | 0.79651 ↓ | −0.32824 |

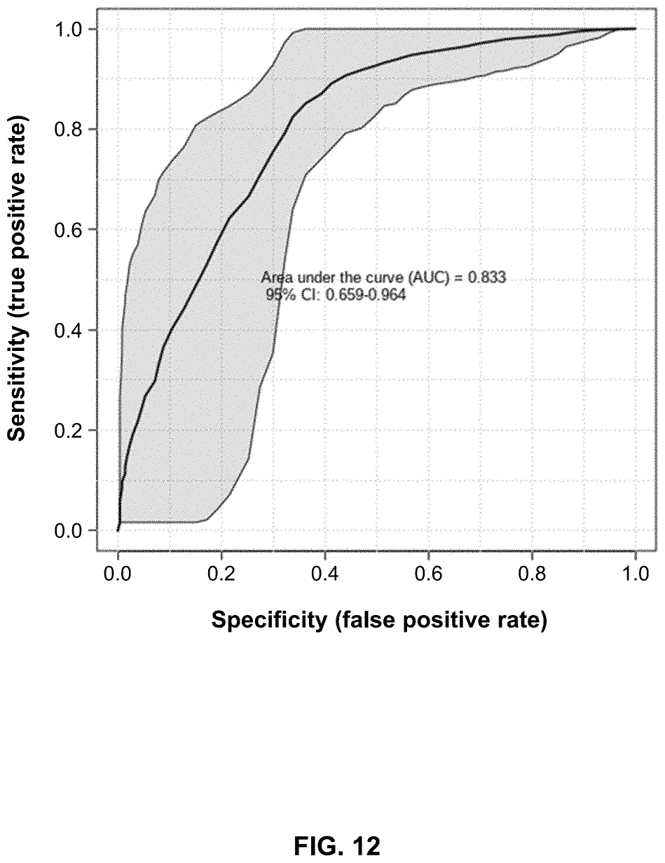

A lipid panel comprising at least LPA 18:0, LPA 16:0, LP C202, CER 240, and LPI 16:1 demonstrated predictability of tumor recurrence in subjects, yielding an AUC>0.83 (see FIG. 12; Table 11).

TABLE 11
Predictive accuracy of five-lipid panel regarding tumor recurrence.

| | | | Specificity^{a} | Sensitivity^{b} |
|---|---|---|---|---|
| Lipids | AUC | 95% CI | (%) | (%) |
| LPA 18:0 | 0.833 | 0.659-0.964 | 44.9 | 27.6 |
| LPA 16:0 | | | | |
| LPC 202 | | | | |
| CER 240 | | | | |
| LPI 16:1 | | | | |

^{a}Specificity at 95% sensitivity
^{b}Sensitivity at 95% specificity

Other lipids were identified as exhibiting a fold change between the two comparative groups and could be included in a panel to predict those who are at risk of tumor recurrence (see Table 12).

TABLE 12
Lipids exhibiting a fold change between subjects who reported
tumor recurrence as compared to those who remained cancer free.

| | | | Fold | Log2 |
|---|---|---|---|---|
| Lipids | p-value | FDR | Change | (FC) |
| LPA 18:0 | 2.26E−08 | 1.34E−05 | 2.7832 ↑ | 1.4767 |
| LPA 18:1 | 3.71E−08 | 1.34E−05 | 4.7793 ↑ | 2.2568 |
| LPA 16:0 | 5.34E−08 | 1.34E−05 | 4.4924 ↑ | 2.1675 |
| LPA 18:2 | 3.33E−07 | 4.53E−05 | 4.5953 ↑ | 2.2002 |
| LPC 202 | 3.61E−07 | 4.53E−05 | 1.7611 ↑ | 0.81648 |
| CER 240 | 3.62E−07 | 4.53E−05 | 0.64043 ↓ | −0.64288 |
| LPC 203 | 7.05E−07 | 7.56E−05 | 1.8265 ↑ | 0.86912 |
| LPC 200 | 9.11E−07 | 8.55E−05 | 1.7954 ↑ | 0.8443 |
| LPI 16:1 | 1.25E−06 | 0.000104 | 0.63072 ↓ | −0.66493 |
| LPA 16:1 | 1.51E−06 | 0.00011 | 3.4713 ↑ | 1.7955 |
| LPC 220 | 1.62E−06 | 0.00011 | 1.9763 ↑ | 0.98282 |
| LPC 201 | 2.03E−06 | 0.000127 | 1.835 ↑ | 0.87581 |
| PS 18:0-18:1 | 3.25E−06 | 0.000186 | 0.57901 ↓ | −0.78834 |
| LPE 220 | 3.48E−06 | 0.000186 | 2.1263 ↑ | 1.0884 |
| LPC 225 | 5.67E−06 | 0.000284 | 1.8262 ↑ | 0.86883 |
| PG 18:0-20:4 | 7.33E−06 | 0.000339 | 0.63786 ↓ | −0.64869 |
| PS 16:1-18:2:2 | 7.67E−06 | 0.000339 | 0.65637 ↓ | −0.60741 |
| PS 16:1-18:1:2 | 8.49E−06 | 0.000354 | 0.68128 ↓ | −0.55368 |
| PG 18:0-20:4:2 | 1.31E−05 | 0.000498 | 0.63517 ↓ | −0.65479 |
| LPC 170 | 1.33E−05 | 0.000498 | 1.6586 ↑ | 0.72999 |
| CE 160 | 1.4E−05 | 0.0005 | 0.60541 ↓ | −0.72402 |
| LPC 241 | 1.65E−05 | 0.000562 | 1.919 ↑ | 0.94039 |
| PS 16:0-18:2 | 1.83E−05 | 0.000597 | 0.70309 ↓ | −0.50822 |
| LPE 161 | 2.15E−05 | 0.000651 | 4.2997 ↑ | 2.1042 |
| LPC 240 | 2.17E−05 | 0.000651 | 1.7351 ↑ | 0.79502 |
| CER 140 | 3.55E−05 | 0.001025 | 0.6886 ↓ | −0.53827 |
| PG 16:0-18:0:2 | 3.91E−05 | 0.001086 | 0.70315 ↓ | −0.50809 |
| PS 18:1-18:2:2 | 4.22E−05 | 0.001101 | 0.68836 ↓ | −0.53876 |
| LPE 240 | 4.25E−05 | 0.001101 | 2.0649 ↑ | 1.046 |
| PS 16:0-16:1:2 | 4.66E−05 | 0.001166 | 0.69582 ↓ | −0.52321 |
| PS 18:0-18:1:2 | 4.95E−05 | 0.001199 | 0.67025 ↓ | −0.57723 |
| PG 16:0-18:0 | 5.72E−05 | 0.001342 | 0.67099 ↓ | −0.57564 |
| PG 16:0-18:1 | 7.01E−05 | 0.001573 | 0.73542 ↓ | −0.44336 |
| PG 16:0-18:1:2 | 7.19E−05 | 0.001573 | 0.73659 ↓ | −0.44107 |
| PS 18:0-18:2 | 7.33E−05 | 0.001573 | 0.68112 ↓ | −0.55402 |
| LPC 204 | 7.84E−05 | 0.00163 | 1.6419 ↑ | 0.71541 |
| LPC 161 | 8.03E−05 | 0.00163 | 1.6078 ↑ | 0.68513 |
| PC 160/160 | 8.37E−05 | 0.001655 | 1.3952 ↑ | 0.48042 |
| PS 18:0-18:0 | 8.91E−05 | 0.001667 | 0.65566 ↓ | −0.60898 |
| PS 16:1-18:0:2 | 9.33E−05 | 0.001667 | 0.67334 ↓ | −0.57059 |
| LPC 226 | 9.46E−05 | 0.001667 | 1.7623 ↑ | 0.81748 |
| PG 18:0-18:1 | 9.78E−05 | 0.001667 | 0.7308 ↓ | −0.45246 |
| CE 224 | 9.94E−05 | 0.001667 | 0.67876 ↓ | −0.55902 |
| PG 18:0-18:1:2 | 9.94E−05 | 0.001667 | 0.73041 ↓ | −0.45323 |
| PA 18:0-20:3:2 | 0.000101 | 0.001667 | 0.77428 ↓ | −0.36907 |
| PG 18:0-18:2 | 0.000103 | 0.001667 | 0.74473 ↓ | −0.42521 |
| LPC 150 | 0.000104 | 0.001667 | 1.4974 ↑ | 0.58245 |
| PC 140/182 | 0.000118 | 0.001836 | 1.6273 ↑ | 0.7025 |
| CE 181 | 0.00012 | 0.001836 | 0.68543 ↓ | −0.54493 |
| PA 18:1-18:2 | 0.000123 | 0.001844 | 0.72697 ↓ | −0.46003 |
| PG 16:0-20:3:2 | 0.000141 | 0.002045 | 0.7786 ↓ | −0.36105 |
| PG 18:0-18:2:2 | 0.000142 | 0.002045 | 0.74968 ↓ | −0.41565 |
| CE 170 | 0.000146 | 0.002064 | 0.70134 ↓ | −0.51181 |
| PG 18:0-18:0 | 0.000156 | 0.002172 | 0.73619 ↓ | −0.44185 |
| PG 16:0-20:2:2 | 0.000161 | 0.002195 | 0.77323 ↓ | −0.37102 |
| PS 16:0-20:3 | 0.000174 | 0.002336 | 0.72523 ↓ | −0.46348 |
| FFA 182 | 0.000204 | 0.002692 | 0.70241 ↓ | −0.50961 |
| FFA 180 | 0.000232 | 0.003 | 0.70589 ↓ | −0.50249 |
| PG 18:1-18:1 | 0.000255 | 0.003245 | 0.72202 ↓ | −0.46989 |
| CE 180 | 0.000259 | 0.003247 | 0.70017 ↓ | −0.51422 |
| PG 16:1-18:1 | 0.000269 | 0.003307 | 0.71083 ↓ | −0.49242 |
| PS 16:0-18:0 | 0.000277 | 0.003355 | 0.68204 ↓ | −0.55207 |
| PA 18:0-18:2 | 0.000297 | 0.003495 | 0.7575 ↓ | −0.40068 |
| PG 18:1-18:2:2 | 0.000298 | 0.003495 | 0.74518 ↓ | −0.42434 |
| PS 16:0-18:1:2 | 0.000317 | 0.003665 | 0.76963 ↓ | −0.37776 |
| LPC 181 | 0.000338 | 0.003844 | 1.388 ↑ | 0.47298 |
| PG 16:0-18:2:2 | 0.000362 | 0.004055 | 0.8411 ↓ | −0.24965 |
| PEP-181/204 | 0.000391 | 0.004317 | 0.7284 ↓ | −0.45721 |
| HCER 180 | 0.000397 | 0.004321 | 1.8937 ↑ | 0.92123 |
| Cholesterol | 0.000413 | 0.004426 | 0.67963 ↓ | −0.55717 |
| CE 183 | 0.000423 | 0.004473 | 0.63145 ↓ | −0.66327 |
| LPC 140 | 0.00043 | 0.004485 | 1.6639 ↑ | 0.73454 |
| PG 16:0-18:2 | 0.000457 | 0.004698 | 0.82686 ↓ | −0.27428 |
| PG 16:0-20:2 | 0.000478 | 0.004849 | 0.77149 ↓ | −0.37428 |
| PG 16:0-20:3 | 0.000626 | 0.006264 | 0.79452 ↓ | −0.33185 |
| DAG 161/182 | 0.000655 | 0.006414 | 1.5221 ↑ | 0.60607 |
| PG 16:0-16:0 | 0.000661 | 0.006414 | 0.74225 ↓ | −0.43002 |
| PE 182/202 | 0.000669 | 0.006414 | 0.7492 ↓ | −0.41658 |
| PG 18:1-18:2 | 0.000675 | 0.006414 | 0.73762 ↓ | −0.43906 |
| LPE 183 | 0.000694 | 0.006517 | 2.0874 ↑ | 1.0617 |
| LPC 221 | 0.000794 | 0.00736 | 2.0573 ↑ | 1.0408 |
| PEP 180/204 | 0.000863 | 0.007907 | 0.72338 ↓ | −0.46718 |
| CE 182 | 0.000988 | 0.008941 | 0.64154 ↓ | −0.64038 |
| PG 18:2-18:2 | 0.001032 | 0.009142 | 0.7396 ↓ | −0.43518 |
| PS 16:1-18:0 | 0.001035 | 0.009142 | 0.69403 ↓ | −0.52692 |
| HCER 200 | 0.001171 | 0.010223 | 1.8456 ↑ | 0.88409 |
| FFA 181 | 0.001201 | 0.010364 | 0.7347 ↓ | −0.44477 |
| PG 16:1-18:0:2 | 0.00123 | 0.010495 | 0.70728 ↓ | −0.49964 |
| LPC 182 | 0.001332 | 0.011241 | 1.3318 ↑ | 0.4134 |
| FFA 160 | 0.001496 | 0.012416 | 0.73482 ↓ | −0.44453 |
| PS 16:0-18:0:2 | 0.001505 | 0.012416 | 0.70918 ↓ | −0.49578 |
| CE 204 | 0.001547 | 0.012607 | 0.60872 ↓ | −0.71615 |
| PC 160/140 | 0.001561 | 0.012607 | 1.5759 ↑ | 0.65613 |
| CE 225 | 0.001598 | 0.012769 | 0.61691 ↓ | −0.69686 |
| LCER 220 | 0.00162 | 0.012804 | 0.83129 ↓ | −0.26657 |
| PC 140/161 | 0.001703 | 0.013322 | 1.623 ↑ | 0.6987 |
| CER 220 | 0.001751 | 0.013558 | 0.79601 ↓ | −0.32914 |
| PG 16:1-18:0 | 0.001807 | 0.013851 | 0.72781 ↓ | −0.45837 |
| CER 241 | 0.001868 | 0.014168 | 0.82422 ↓ | −0.27889 |
| PC 140/181 | 0.001898 | 0.014255 | 1.4596 ↑ | 0.5456 |
| SM d18:1-12:0 | 0.002043 | 0.015193 | 1.5362 ↑ | 0.61933 |
| PEP 181/226 | 0.002084 | 0.015347 | 0.80008 ↓ | −0.32179 |
| FFA 222 | 0.002148 | 0.015664 | 0.74505 ↓ | −0.42459 |
| PE 160/160 | 0.002197 | 0.015863 | 2.4598 ↑ | 1.2985 |
| PI 14:0-18:1 | 0.002386 | 0.017063 | 0.76688 ↓ | −0.38292 |
| LPC 183 | 0.002435 | 0.017248 | 1.4718 ↑ | 0.55762 |
| PEP 80/226 | 0.002571 | 0.018008 | 0.79804 ↓ | −0.32546 |
| LPC 180 | 0.00259 | 0.018008 | 1.2124 ↑ | 0.27787 |
| FFA 120 | 0.002987 | 0.020578 | 0.72057 ↓ | −0.47279 |
| PC 120/182 | 0.00309 | 0.021098 | 1.438 ↑ | 0.52407 |
| PG 16:1-18:2:2 | 0.003126 | 0.021149 | 0.8832 ↓ | −0.17918 |
| FFA 204 | 0.003204 | 0.021483 | 0.76755 ↓ | −0.38166 |
| PG 16:1-18:1:2 | 0.003322 | 0.022075 | 0.84619 ↓ | −0.24095 |
| CE 226 | 0.003712 | 0.024452 | 0.66706 ↓ | −0.58411 |
| PC 180/140 | 0.004024 | 0.026277 | 1.5577 ↑ | 0.63942 |
| FFA 170 | 0.004063 | 0.026302 | 0.75367 ↓ | −0.40799 |
| PC 140/183 | 0.00421 | 0.026958 | 1.5771 ↑ | 0.65731 |
| CE 203 | 0.004236 | 0.026958 | 0.71444 ↓ | −0.48511 |
| SM d18:1-14:1 | 0.004573 | 0.028859 | 1.3414 ↑ | 0.42371 |
| PEP 182/204 | 0.005086 | 0.031827 | 0.76879 ↓ | −0.37934 |
| PEP 181/225 | 0.005343 | 0.033161 | 0.82329 ↓ | −0.28053 |
| FFA 140 | 0.005395 | 0.033208 | 0.77927 ↓ | −0.3598 |
| PE 180/150 | 0.005535 | 0.033771 | 2.035 ↑ | 1.025 |
| HCER 201 | 0.005576 | 0.033771 | 1.7378 ↑ | 0.7973 |
| PC 140/140 | 0.005717 | 0.034349 | 1.7486 ↑ | 0.80623 |
| TAG 522-FA 160 | 0.006008 | 0.035812 | 0.81083 ↓ | −0.30254 |
| PEP 160/226 | 0.006428 | 0.038012 | 0.81902 ↓ | −0.28804 |
| LPC 205 | 0.006703 | 0.038843 | 1.9347 ↑ | 0.9521 |
| PG 16:0-16:1:2 | 0.006723 | 0.038843 | 0.81805 ↓ | −0.28973 |
| FFA 184 | 0.006724 | 0.038843 | 0.74607 ↓ | −0.42261 |
| PEP 182/226 | 0.006903 | 0.039572 | 0.77989 ↓ | −0.35865 |
| FFA 200 | 0.007206 | 0.040997 | 0.73183 ↓ | −0.45043 |
| PEP 160/161 | 0.008289 | 0.046803 | 1.837 ↑ | 0.87737 |
| FFA 150 | 0.008367 | 0.04689 | 0.7372 ↓ | −0.43987 |
| PC 180/180 | 0.008697 | 0.048382 | 1.3671 ↑ | 0.45108 |
| HCER 140 | 0.008827 | 0.04874 | 1.6286 ↑ | 0.70366 |

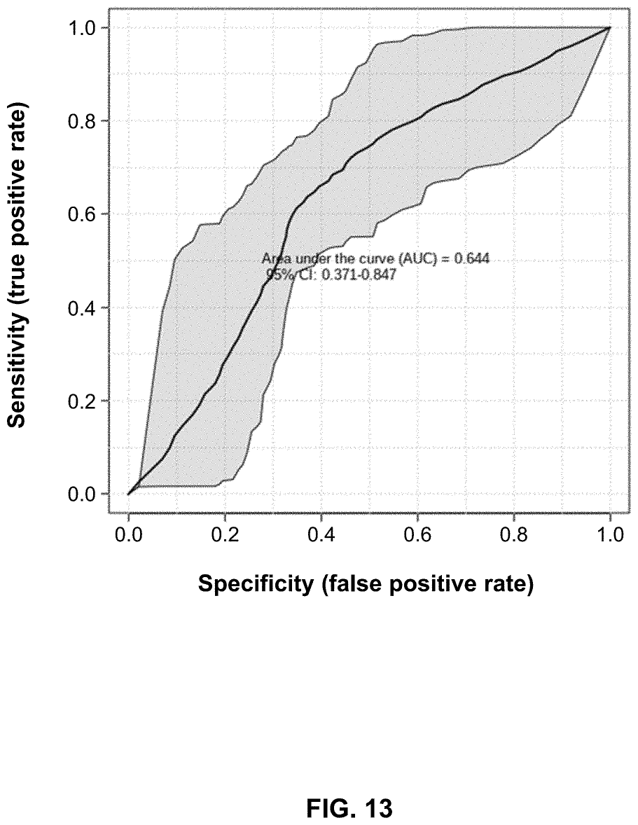

Moreover, a lipid panel comprising at least LPA 18:0, DAG 160/781, LPA 16:0, and DAG 181/181 yielded an AUC >0.64 (see FIG. 13; Table 13).

TABLE 13
Predictive accuracy of four-lipid panel regarding late effects.

| | | | Specificity^{a} | Sensitivity^{b} |
|---|---|---|---|---|
| Lipids | AUC | 95% CI | (%) | (%) |
| LPA 18:0 | 0.644 | 0.371-0.847 | 7.6 | 12.9 |
| DAG 160/18 | | | | |
| LPA 16:0 | | | | |
| DAG 181/181 | | | | |

^{a}Specificity at 95% sensitivity
^{b}Sensitivity at 95% specificity

Other lipids were identified as exhibiting a fold change between the two comparative groups and could be included in a panel to predict those who are at risk of late effects (see Table 14).

TABLE 14
Lipids exhibiting a fold change between subjects who reported late
effects as compared to those who did not exhibit late effects.

| | | | Fold | Log2 |
|---|---|---|---|---|
| Lipids | p-value | FDR | Change | (FC) |
| LPA 18:0 | 0.001477 | 0.81883 | 1.499 ↑ | 0.58401 |
| DAG 160/181 | 0.002459 | 0.81883 | 0.76898 ↓ | −0.37899 |
| LPA 16:0 | 0.003271 | 0.81883 | 1.511 ↑ | 0.59548 |
| DAG 181/181 | 0.014358 | 0.9951 | 0.81241 ↓ | −0.29971 |
| LPI 16:0 | 0.01766 | 0.9951 | 0.77799 ↓ | −0.36218 |
| LPA 18:2 | 0.026652 | 0.9951 | 1.3924 ↑ | 0.47754 |
| LPA 18:1 | 0.029963 | 0.9951 | 1.3063 ↑ | 0.38546 |
| PI 16:1-18:1:2 | 0.030236 | 0.9951 | 0.63228 ↓ | −0.66136 |
| CE201 | 0.039263 | 0.9951 | 1.4493 ↑ | 0.53531 |
| LPA 16:1 | 0.042455 | 0.9951 | 1.2295 ↑ | 0.29802 |
| FFA 202 | 0.043376 | 0.9951 | 1.4351 ↑ | 0.52115 |
| PC 180/225 | 0.043516 | 0.9951 | 0.85141 ↓ | −0.23208 |
| PE 180/225 | 0.049385 | 0.9951 | 0.84731 ↓ | −0.23904 |

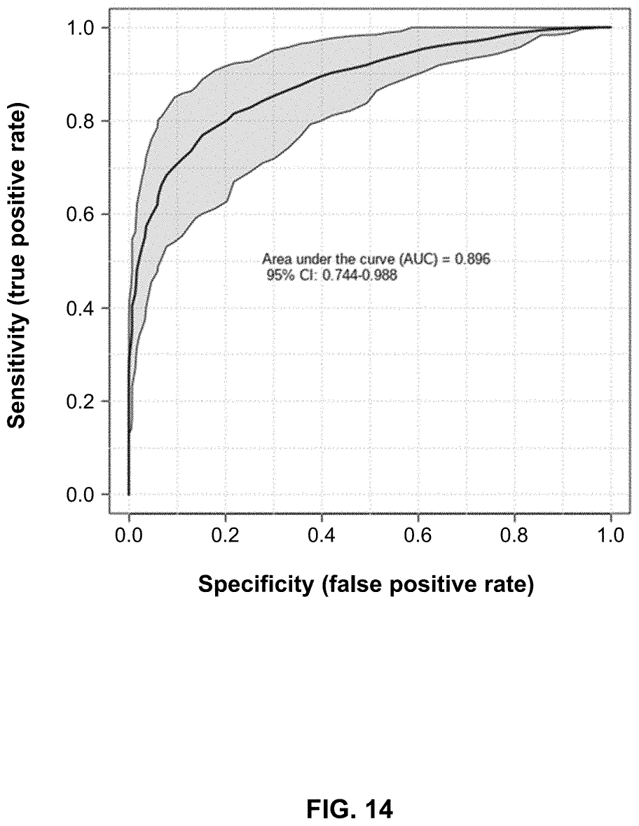

Further, panels that combine metabolites and lipid exhibited predictability of tumor recurrence and late effects in subjects. For example, a panel comprising at least metabolites geranyl pyrophosphate, glucose 1-phosphate, and 3-hydroxy-3-methylglutaryl-CoA, and at least lipids LPA 18:0, LPA 16:0, LPC 202, CER 240, and LPI 16:1, demonstrated predictability of tumor recurrence in subjects, yielding an AUC>0.89 (see FIG. 14; Tables 15 and 16).

TABLE 15
Predictive accuracy of three-metabolite,
five- panel regarding late effects.

| | | | Specificity^{a} | Sensitivity^{b} |
|---|---|---|---|---|
| Metabolites/Lipids | AUC | 95% CI | (%) | (%) |
| Geranyl pyrophosphate | 0.896 | 0.744-0.988 | 41.2 | 76.4 |
| Glucose 1-phosphate | | | | |
| 3-hydroxy-3- | | | | |
| methylglutaryl-CoA | | | | |
| LPA 18_0 | | | | |
| LPA 16_0 | | | | |
| LPC202 | | | | |
| CER240 | | | | |
| LPI 16_1 | | | | |

^{a}Specificity at 95% sensitivity
^{b}Sensitivity at 95% specificity

TABLE 16
Metabolites and lipids exhibiting a fold change
between subjects who reported late effects as
compared to those who remained cancer free.

| | | | Fold | Log2 |
|---|---|---|---|---|
| Metabolites/Lipids | p-value | FDR | Change | (FC) |
| Geranyl | 2.89E−06 | 2.05E−04 | 1.80 ↑ | 0.85 |
| pyrophosphate | | | | |
| Glucose 1- | 1.64E−05 | 7.75E−04 | 2.39 ↑ | 1.25 |
| phosphate | | | | |
| 3-hydroxy-3- | 3.55E−05 | 8.41E−04 | 1.86 ↑ | 0.89 |
| methylglutaryl-CoA | | | | |
| LPA 18:0 | 2.26E−08 | 1.34E−05 | 2.7832 ↑ | 1.4767 |
| LPA 16:0 | 5.34E−08 | 1.34E−05 | 4.4924 ↑ | 2.1675 |
| LPC 202 | 3.61E−07 | 4.53E−05 | 1.7611 ↑ | 0.81648 |
| CER 240 | 3.62E−07 | 4.53E−05 | 0.64043 ↓ | −0.64288 |
| LPI 16:1 | 1.25E−06 | 0.000104 | 0.63072 ↓ | −0.66493 |

A panel comprising at least metabolites metanephrine, tryptophan, xanthurenic acid, and pantothenate, and at least lipids LPA 18:0, DAG 160/181, LPA 16:0, and DAG 181/181 yielded an AUC>0.64 (see FIG. 14; Tables 17 and 18).

TABLE 17
Predictive accuracy of four-metabolite,
four-lipid panel regarding late effects.

| | | | Specificity^{a} | Sensitivity^{b} |
|---|---|---|---|---|
| Metabolites/Lipids | AUC | 95% CI | (%) | (%) |
| Metanephrine | 0.644 | 0.371-0.847 | 16.5 | 5.4 |
| Tryptophan | | | | |
| Xanthurenic acid | | | | |
| Pantothenate | | | | |
| LPA 18_0 | | | | |
| DAG160/181 | | | | |
| LPA 16_0 | | | | |
| DAG181/181 | | | | |

^{a}Specificity at 95% sensitivity
^{b}Sensitivity at 95% specificity

TABLE 18
Metabolites and lipids exhibiting a fold change
between subjects who reported late effects as
compared to those who remained cancer free.

| | | | Fold | Log2 |
|---|---|---|---|---|
| Metabolites | p-value | FDR | Change | (FC) |
| Metanephrine | 0.002627 | 0.33057 | 0.36389 ↓ | −1.4584 |
| Tryptophan | 0.004656 | 0.33057 | 0.64815 ↓ | −0.6256 |
| Xanthurenic acid | 0.007601 | 0.33885 | 0.6962 ↓ | −0.52242 |
| Pantothenate | 0.010219 | 0.33885 | 0.64877 ↓ | −0.62421 |
| LPA 18:0 | 0.001477 | 0.81883 | 1.499 ↑ | 0.58401 |
| DAG 160/181 | 0.002459 | 0.81883 | 0.76898 ↓ | −0.37899 |
| LPA 16:0 | 0.003271 | 0.81883 | 1.511 ↑ | 0.59548 |
| DAG 181/181 | 0.014358 | 0.9951 | 0.81241 ↓ | −0.29971 |

What is claimed is:

1. A method of treating with radiation therapy a subject having cancer, the method comprising administering radiation therapy to the subject, wherein the subject does not have an increased risk of having an adverse reaction to radiation therapy,
wherein the subject has an increased risk of an adverse reaction to radiation therapy when the subject's level of each component in a component profile from a sample of the subject is altered as compared to a normal level of each component.

2. The method of claim 1, wherein the adverse reaction is tumor recurrence.

3. The method of claim 2, wherein the component profile comprises geranyl pyrophosphate, glucose-1-phosphate, and 3-hydroxy-3-methylglutaryl-CoA.

4. The method of claim 3, wherein the subject's level of each component is altered as compared to the normal level of each component when the subject's level of geranyl pyrophosphate, glucose-1-phosphate, and 3-hydroxy-3-methylglutaryl-CoA is higher as compared to the normal level of geranyl pyrophosphate, glucose-1-phosphate, and 3-hydroxy-3-methylglutaryl-CoA, respectively.

5. The method of claim 2, wherein the component profile comprises LPA 18:0, LPA 16:0, LPC 20:2, CER 24:0, and LPI 16:1.

6. The method of claim 5, wherein the subject's level of each component is altered as compared to the normal level of each component when the subject's level of LPA 18:0, LPA 16:0, and LPC 20:2 is higher than the normal level of LPA 18:0, LPA 16:0, and LPC 20:2, respectively; and the subject's level of CER 24:0 and LPI 16:1 is lower than the normal level of CER 24:0 and LPI 16:1, respectively.

7. The method of claim 2, wherein the component profile comprises geranyl pyrophosphate, glucose-1-phosphate, 3-hydroxy-3-methylglutaryl-CoA, LPA 18:0, LPA 16:0, LPC 20:2, CER 24:0, and LPI 16:1.

8. The method of claim 7, wherein the subject's level of each component is altered as compared to the normal level of each component when the subject's level of geranyl pyrophosphate, glucose-1-phosphate, 3-hydroxy-3-methylglutaryl-CoA, LPA 18:0, LPA 16:0, and LPC 20:2 is higher as compared to the normal level of geranyl pyrophosphate, glucose-1-phosphate, 3-hydroxy-3-methylglutaryl-CoA, LPA 18:0, LPA 16:0, and LPC 20:2, respectively;
and the subject's level of CER 24:0 and LPI 16:1 is lower than the normal level of CER 24:0 and LPI 16:1, respectively.

9. The method of claim 1, wherein the adverse reaction is one or more late effects.

10. The method of claim 9, wherein the late effects comprise rectal toxicity, urinary toxicity, or a combination thereof.

11. The method of claim 9, wherein the component profile comprises metanephrine, tryptophan, xanthurenic acid, and pantothenate.

12. The method of claim 11, wherein the subject's level of each component is altered as compared to the normal level of each component when the subject's level of metanephrine, tryptophan, xanthurenic acid, and pantothenate is lower than the normal level of metanephrine, tryptophan, xanthurenic acid, and pantothenate, respectively.

13. The method of claim 9, wherein the component profile comprises LPA 18:0, DAG 16:0/18:0, LPA 16:0, and DAG 18:1/18:1.

14. The method of claim 13, wherein the subject's level of each component is altered as compared to the normal level of each component when the subject's level of LPA18:0 and LPA 16:0 is higher than the normal level of LPA18:0 and LPA 16:0, respectively; and the subject's level of DAG 16:0/18:1 and DAG 18:1/18:1 is lower than the normal level of DAG 16:0/18:1 and DAG 18:1/18:1, respectively.

15. The method of claim 9, wherein the component profile comprises metanephrine, tryptophan, xanthurenic acid, pantothenate, LPA 18:0, DAG 16:0/18:0, LPA 16:0, and DAG 18:1/18:1.

16. The method of claim 15, wherein the subject's level of each component is altered as compared to the normal level of each component when the subject's level of LPA18:0 and LPA 16:0 is higher than the normal level of LPA18:0 and LPA 16:0, respectively; and the subject's level of metanephrine, tryptophan, xanthurenic acid, pantothenate, DAG 16:0/18:1, and DAG 18:1/18:1 is lower than the normal level of metanephrine, tryptophan, xanthurenic acid, pantothenate, DAG 16:0/18:1, and DAG 18:1/18:1, respectively.

17. The method of claim 1, wherein the normal level of the component comprises the subject's level of the component prior to receiving radiation treatment for cancer.

18. The method of claim 1, wherein the normal level of the component comprises a level generated from a population of individuals that did not have an adverse reaction after receiving radiation treatment for cancer.

19. A method of measuring levels of components in a component profile in a subject, the method comprising determining the level of each of the components, wherein the component profile comprises the component profile of claim 7.

20. A method of measuring levels of components in a component profile in a subject, the method comprising determining the level of each of the components, wherein the component profile comprises the component profile of claim 15.
